(19) United States
(12) Patent Application Publication
(10) Pub. No.: US 2007/0166366 A1
(43) Pub. Date: Jul. 19, 2007

(54) Autologous upregulation mechanism allowing optimized cell type-specific and regulated gene expression cells

(76) Inventors: Jian-Yun Dong, Mt. Pleasant, SC (US); Semyon Rubinchik, Mr. Pleasant, SC (US); Jan Woraratanadharm, Mount Pleasant, SC (US)

Correspondence Address: FULBRIGHT & JAWORSKI L.L.P., 600 CONGRESS AVE., SUITE 2400, AUSTIN, TX, 78701, US

(21) Appl. No.: 10/555,109

(22) PCT Filed: Apr. 30, 2004
(86) PCT No.: PCT/US04/13487

§ 371 Date: Dec. 13, 2006

Related U.S. Application Data
(60) Provisional application No. 60/467,171, filed on May 1, 2003.

(51) Int. Cl. A61K 48/00; A61K 9/127; C12N 15/88

(52) U.S. Cl. 424/450; 514/44; 435/458

(57) ABSTRACT

The present invention provides methods for high level, regulated transgene transcription that is restricted to cell populations of specific types. The process is designed to work with any inducible expression regulation systems, adapting them to a tissue-specific expression pattern while simultaneously delivering maximal achievable expression levels. In particular, the invention utilizes hybrid promoters that contain the DNA elements for both cell type-specific and regulated transcription. By placing the gene of the transcriptional activation factor (TAF) under the control of this tissue-specific/drug-regulated (TSDR) promoter, this invention achieves high expression levels of TAF in specific target cells by first initiating TAF expression using cell-type specific transcription elements, and subsequently amplifying transcriptional activity by establishing an autoregulatory positive feedback loop. In non-target cells, cell type-specific elements of the TSDR promoter will be inactive, the TAF expression will not be initiated, and auto-upregulation will not occur. For cell type-specific promoters with leaky low-level activity in non-target cells, a variation of this system has been developed which combines autologous upregulation of TAF with the expression of cross-competing transcriptional silencers (TSi) to achieve a type of eukaryotic “genetic switch”—either shutting off transgene and TAF expression completely or promoting maximal expression levels, depending on the original activity level of the specific promoter in that particular cell.

The present application claims benefit of priority to U.S. Provisional Application Ser. No. 60/467,171, filed May 1, 2003, the entire contents of which are hereby incorporated by reference.

### BACKGROUND OF THE INVENTION

1. Field of the Invention

The present invention relates generally to the fields of molecular biology and gene therapy, and more specifically to the combined spatial and quantitative regulation of transgene expression in eukaryotic cells. In particular, the present invention relates to a system for restricting transgene transcription to specific cell types while at the same time efficiently regulating its expression levels.

2. Description of Related Art

For 2003, it was estimated that 220,900 new cases of prostate cancer would be diagnosed, and 28,900 men would die from this disease. Although the five-year relative survival rate for patients with diagnoses in the local and regional stages is 100%, approximately 30% of patients treated for localized disease relapse (Pound, 1997). In addition, current treatments of localized prostate cancer are not without complications (Meuleman and Mulders, 2003, Hara et al., 2003; Dahm et al., 2003; Kirschner-Hermanns and Jakse, 2002). Radical prostatectomy involves undergoing major surgery and often results in temporary to permanent complications such as incontinence and impotence (Meuleman and Mulders, 2003; Hara et al., 2003; Kirschner-Hermanns and Jakse, 2002). Furthermore, not all cases of local disease can be treated by the traditional local curative approaches due to local invasion of nearby tissues and a loss of differentiation. Locally advanced tumor growth can lead to bladder outlet obstruction, base of bladder invasion, urethral obstruction, and local pain and discomfort in these patients (Klein et al., 2001). Therefore, there is clearly a need to investigate alternative treatment strategies to expand the arsenal of locally advanced prostate cancer treatment options.

One such treatment alternative is the use of gene therapy vectors to specifically eliminate prostate cancer cells utilizing pro-apoptotic genes including Fas ligand (FasL), tumor necrosis factor (TNF)-related apoptosis inducing ligand (TRAIL), or Bax. Because many of these cancer gene therapy strategies involve the induction of a toxic gene product to eliminate the cancer cells, it is important to localize that transgene expression to target cells only. Incorporation of tissue-specific promoters to localize transgene expression has been utilized for several cytotoxic cancer gene therapy vectors that have been investigated in clinical trials (Doehn and Jocham, 2001; Kubo et al., 2003; Shirakawa et al., 2000).

One tissue-specific promoter that has shown promise as a candidate promoter for driving cytotoxic transgenes for the development of a prostate cancer gene therapy vector is the ARR2PB promoter. This synthetically derived, prostate-specific promoter was developed from regulatory elements from the rat probasin promoter (Snoek et al., 1998; Kasper et al., 1999; Zhang et al., 2000). This promoter demonstrated good prostate-specific regulation both in vitro and in transgenic mice (Zhang et al., 2000; Wu et al., 2001; Andriani et al., 2001; Rubinchik et al., 2001). Although the ARR2PB promoter includes two androgen response regions that greatly enhance prostate-specific transgene expression, induced transgene expression from ARR2PB, like that from most mammal-derived tissue-specific promoters, still tends to be significantly weaker than that induced by virus-derived promoters, such as the human cytomegalovirus intermediate/early (hCMVie) promoter (Rubinchik et al., 2001).

Previously attempts have been made to enhance the transcriptional activity of the, ARR2PB promoter by combining the ARR2PB promoter with elements of the tetracycline (Tet) regulatory system (Gossen and Bujard, 1992; Furth et al., 1994; Kistner et al., 1996) in a single adenoviral vector (Rubinchik et al., 2001). While this vector, known as the Ad/FasL-GFP_{PS/TR} vector, was successful in enhancing the induced levels of a Fas ligand-green fluorescent protein (FasL-GFP) fusion protein in prostate cancer cells, this combination of regulatory elements also resulted in a decrease in prostate specificity. This reduction in specificity may have been the result of an inherent limitation of the tetracycline responsive element (TRE) promoter from which some transgene expression still occurs even under uninduced conditions (i.e., presence of excess doxycycline (dox, a tetracycline analog) in the case of the Tet activator (tTA); or the absence of dox in the case of the reverse Tet activator (rtTA)) in transient cell transduction systems like adenovirus. This has been observed by a number of groups (Furth et al., 1994; Kistner et al., 1996; Howe, Jr. et al., 1995). Such leaky expression could be quite detrimental in terms of a cancer gene therapy vector.

In addition to regulatability, there is an increasing recognition of a requirement to restrict transgene expression to appropriate cells and tissues in the organism. This not only applies to the treatment of systemic diseases, such as metastatic cancer, but also to local gene and cancer therapy, whose efficacy and safety can be improved by restricting transgene expression to specific cell populations. Each differentiated cell type has a unique “fingerprint” of transcripts specific to it alone. Although the majority of proteins in that category are found in more than one tissue at various levels of expression, some are uniquely associated with a specific cell type. Similarly, many types of tumor cells overexpress proteins found at low levels in normal cells, or express fetal proteins normally downregulated in cells of an adult organism. For the majority of proteins with cell type-restricted expression pattern, that specificity is controlled at the level of transcription by their promoters, through the use of cell-type specific transcription factors. Many experimental gene therapy protocols currently make use of such promoters to restrict transgene expression to a specific cell population. Frequently, however, specific promoters are inefficient activators of transcription which may limit their applicability.

Clearly, if the transgene expression is not tightly regulated, toxic proteins can be non-specifically expressed in non-target cells, leading to unwanted destruction of non-cancerous tissues. Thus, improved methods of controlled gene delivery are required.

### SUMMARY OF THE INVENTION

Therefore, in accordance with the present invention, there is provided an expression vector comprising (a) a first expression cassette comprising a first coding region that encodes a transcriptional activating factor (TAF), said first coding region being positioned under the transcriptional control of a first promoter comprising (i) a tissue specific regulatory element (TSRE) and (ii) a TAF binding site (TBS); and (b) a second expression cassette comprising a second coding region that encodes a selected polypeptide, said second coding region being positioned under the transcriptional control of a second promoter comprising (i) a TSRE and a TBS or (ii) a TBS. The expression vector may further comprise (c) a third expression cassette comprising a third coding region that encodes a first transcriptional silencer (TSI), said third coding region being positioned under the transcriptional control a third promoter comprising (i) a TSRE and (ii) a TAB; and (d) a fourth expression cassette comprising a fourth coding region that encodes a second TSI, said fourth coding region being positioned under the transcriptional control of a fourth promoter that is negatively regulated by said first TSI, wherein said first, second and third promoters are negatively regulated by said second TSI.

The expression vector may be a non-viral vector, for example, one comprised within a lipid delivery vehicle such as a a liposome. The expression vector may also be a viral vector, such as an adenoviral vector, a retroviral vector, a herpesviral vector, a pox virus vector, a polyoma virus vector, an alpha virus vector, or an adeno-associate viral vector. The viral vector may be comprised within a viral particle. The viral vector may be a replication-deficient viral vector, such as a replication-deficient adenoviral vector, or a replication-competent viral vector or a conditionally replication-competent viral vector, such as a replication-competent or conditionally replication competent adenoviral vector. The vector may further comprise a selectable or screenable marker.

The TAF may be an antibiotic-regulated TAF, a hormone-regulated TAF, an human immunodeficiency virus TAF, or a hepatocye TAF (e.g., HNF-1). The TSRE may be derived from an ARR2PB promoter, a probasin promoter, an osteocalcin promoter, a human kallikrein 2 promoter, a DD3 promoter, a Clara cell secretory protein promoter, a liver-type pyruvate kinase proximal promoter, an apoE promoter, an alcohol dehydrogenase 6 promoter, a MUC-1 promoter, a survivin promoter, a CCR5 promoter a PSA promoter, an AFP promoter, an albumin promoter, or a telomerase promoter. The selected polypeptide may be a therapeutic polypeptide, such as an anti-cancer polypeptide (e.g., tumor suppressor, and inducer of apoptosis, and cell cycle regulator, a toxin, or an inhibitor of angiogenesis), an enzyme, a cytokine, a hormone, a tumor antigen, a human antigen or a pathogen antigen. The selected polypeptide is essential for vector replication, for example, where said vector is an adenoviral vector, said selected polypeptide may be an E1 protein, and E2 protein, an E4 protein, a fiber capside protein, an adenovirus terminal binding protein, an adenovirus polymerase. Where said vector is a herpes simplex virus, said selected polypeptide may be a herpes simplex virus early or late gene.

Another embodiment, there is provided a method of expressing a selected polypeptide in a cell of interest comprising contacting said cell with an expression vector comprising (a) a first expression cassette comprising a first coding region that encodes a transcriptional activating factor (TAF), said first coding region being positioned under the transcriptional control of a first promoter comprising (i) a tissue specific regulatory element (TSRE) and (ii) a TAF binding site (TBS); and (b) a second expression cassette comprising a second coding region that encodes a selected polypeptide, said second coding region being positioned under the transcriptional control of a second promoter comprising (i) a TSRE and a TBS or (ii) a TBS. The expression vector may further comprise (c) a third expression cassette comprising a third coding region that encodes a first transcriptional silencer (TSI), said third coding region being positioned under the transcriptional control a third promoter comprising (i) a TSRE and (ii) a TAB; and (d) a fourth expression cassette comprising a fourth coding region that encodes a second TSI, said fourth coding region being positioned under the transcriptional control of a fourth promoter that is negatively regulated by said first TSI, wherein said first, second and third promoters are negatively regulated by said second TSI.

The vector may be a non-viral vector or a viral vector, such as an adenoviral vector, a retroviral vector, a herpesviral vector, a pox virus vector, a polyoma virus vector, an alpha virus vector or an adeno-associate viral vector. The viral vector may be a replication-deficient viral vector, a replication-competent viral vector, or a conditionally replication-competent viral vector. The TAF may be an antibiotic-regulated TAF, a hormone-regulated TAF, an human immunodeficiency virus TAF, or a hepatocye TAF. The TSRE may be derived from an ARR2PB promoter, a probasin promoter, an osteocalcin promoter, a human kallikrein 2 promoter, a DD3 promoter, a Clara cell secretory protein promoter, a liver-type pyruvate kinase proximal promoter, an apoE promoter, an alcohol dehydrogenase 6 promoter, a MUC-1 promoter, a survivin promoter, a CCR5 promoter a PSA promoter, an AFP promoter, an albumin promoter, or a telomerase promoter. The vector may further comprise a selectable or screenable marker.

In yet another embodiment, there is provided a method of treating cancer comprising administering to a subject having cancer an expression vector comprising (a) a first expression cassette comprising a first coding region that encodes a transcriptional activating factor (TAF), said first coding region being positioned under the transcriptional control of a first promoter comprising (i) a tissue specific regulatory element (TSRE) and (ii) a TAP binding site (TBS); and (b) a second expression cassette comprising a second coding region that encodes a selected polypeptide, said second coding region being positioned under the transcriptional control of a second promoter comprising (i) a TSRE and a TBS or (ii) a TBS. The expression vector may further comprise (c) a third expression cassette comprising a third coding region that encodes a first transcriptional silencer (TSI), said third coding region being positioned under the transcriptional control a third promoter comprising (i) a TSRE and (ii) a TAB; and (d) a fourth expression cassette comprising a fourth coding region that encodes a second TSI, said fourth coding region being positioned under the transcriptional control of a fourth promoter that is negatively regulated by said first TSI, wherein said first, second and third promoters are negatively regulated by said second TSI.

The vector may be a non-viral vector or a viral vector, such as an adenoviral vector, a retroviral vector, a herpesviral vector, a pox virus vector, a polyoma virus vector, an alpha virus vector or an adeno-associate viral vector. The viral vector may be a replication-deficient viral vector, a replication-competent viral vector, or a conditionally replication-competent viral vector. The TAF may be an antibiotic-regulated TAF, a hormone-regulated TAF, an human, immunodeficiency virus TAF, or a hepatocye TAF. The TSRE may be derived from an ARR2PB promoter, a probasin promoter, an osteocalcin promoter, a human kallikrein 2 promoter, a DD3 promoter, a Clara cell secretory protein promoter, a liver-type pyruvate kinase proximal promoter, an apoE promoter, an alcohol dehydrogenase 6 promoter, a MUC-1 promoter, a survivin promoter, a CCR5 promoter a PSA promoter, an AFP promoter, an albumin promoter, or a telomerase promoter. The vector may further comprise a selectable or screenable marker.

The cancer may be breast cancer, ovarian cancer, fallopian tube cancer, cervical cancer, uterine cancer, prostate cancer, testicular cancer, pancreactic cancer, colon cancer, bladder cancer, liver cancer, stomach cancer, lung cancer, lymphoid cancer, brain cancer, thyroid cancer, head & neck cancer, skin cancer or leukemia. The expression vector may be administered more than once, may be administered intratumorally, into tumor vasculature, local to a tumor, regional to a tumor, systemically, intravenously, intraarterially, subcutaneously, intramuscularly or into a natural or artificial body cavity. The cancer may be a recurrent cancer, a metastatic cancer or a drug resistant cancer. The method may further comprise administering to said subject one or more distinct cancer therapies, such as chemotherapy, radiotherapy, hormonal therapy, immunotherapy, cryotherapy, toxin therapy, surgery or a second gene therapy. The expression vector may be provided to said subject at the same time as said distinct cancer therapy, before said distinct cancer therapy, or after said distinct cancer therapy.

In still yet another embodiment, there is provided an expression vector comprising (a) a first expression cassette comprising a first coding region that encodes a first transcriptional silencer (TSI), said first coding region being positioned under the transcriptional control of a first promoter comprising a TSI binding site (SBS) for a second TSI; (b) a second expression cassette comprising a second coding region that encodes a transcriptional activating factor (TAF), said second coding region being positioned under the transcriptional control of a second promoter comprising a tissue specific regulatory element (TSRE); (c) a third expression cassette comprising a third coding region that encodes said second TSI, said third coding region being positioned under the transcriptional control of a third promoter comprising a tissue specific regulatory element (TSRE); and (d) a fourth expression cassette comprising a fourth coding region that encodes a selected polypeptide, said fourth coding region being positioned under the transcriptional control of a fourth promoter comprising a TAF binding site. Also provided are a method of expressing a selected polypeptide in a cell of interest comprising contacting said cell with this expression vector, and a method of treating cancer comprising administering to a subject having cancer this expression vector. All of the preceding vector and method limitations may be applied to this embodiment as well.

Moreover, all of the preceding expression cassettes may be separated into distinct expression vectors for separate but combined provision to cells or subjects.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—Schematic representation of a generalized autologously upregulated cell-type specific/ligand-inducible gene expression regulation system, in either (FIG. 1A) a specific target cell in the presence (right panel) or the absence (left panel) of inducing ligand, or in a (FIG. 1B) non-specific cell. TAF—transcription activating factor.

FIG. 2—Schematic representation of a generalized autologously upregulated cell-type specific/ligand inducible gene expression regulation system controlled by a cross-inhibiting transcriptional silencer “gene switch” mechanism, in either non-target or target cells.

FIGS. 3A-B—Schematic diagram demonstrating (FIG. 3A) construction of the TRE-ARR2PB hybrid promoter from the elements of the TRE-mCMV and ARR2PB promoters, and (FIG. 3B) structure of the two rAd vectors delivering regulated expression of the GFP reporter, rAd/GFP_{tTA} which utilizes Tet-OFF expression regulation system, and rAd/GFP_{PFLPS} which delivers a prostate-specific expression pattern with a positive feedback loop upregulation of tTA expression.

FIG. 4—Bar graph depicting GFP expression in prostate-derived LNCaP and non-prostate U373MG cells transduced with rAd/GFP_{tTA} and rAd/GFP_{PFLPS} vectors at MOI of 30. Cells were incubated in the presence of 10 nM dihydrotestosterone, which activates androgen receptor function, and with or without doxycycline in culture medium, as indicated. GFP fluorescence in cell lysates from each well was analyzed 48 hours post-transduction by BMG Labtechnologies FluoStar plate reader. Averages and standard deviations of 3 experiments are shown.

FIG. 5—Line graph which demonstrates that GFP expression in LNCaP cells can be regulated by altering the concentration of doxycycline in cell culture medium. Cells were transduced by the rAd/GFP_{PFLPS} vector at MOI of 30 and cultured in the presence of indicated concentrations of dox and 3 nM DHT. GFP fluorescence was determined as described in FIGS. 3A-B. Averages and standard deviations of 3 experiments are shown.

FIGS. 6A-B—(FIG. 6A) A schematic representation of the structure and activity of the LRE promoter. Shown are the positions of the TATA and CAT sequences of hCMVi/e promoter, and the insertion of the lacO sites. Lower panel demonstrates LRE promoter regulation by LacR. 293 cells were co-transfected with a plasmid containing GFP under LRE promoter control and either pLacR plasmid or control vector. Ability to partially regulate LRE activity with IPTG is also demonstrated. (FIG. 6B) Schematic representation of the Gene Switch vector. An expression cassette containing the LRE promoter driving tTS expression was cloned into the left end of the genome, while the complex expression cassette containing ARR2PB driving LacI, ARR2PB driving tTA, and TRE promoter driving GFP was cloned into the right end. Table delineating the results expected following vector transduction of prostate cells versus non-prostate cells.

FIG. 7—Demonstration of the cell-type specific regulation achieved with the cross-inhibiting transcriptional silencers. Prostate cancer cells (LNCaP) and non-prostate cells (U251MG) were transduced with Ad/CMV.GFP, Ad/GFP_{tTA}(TET), Ad/GFP_{DiSTRES} (PSTRGS), or Ad/CMV.LacZ as control at MOI 100 and cultured in the presence of 30 nM DHT. GFP fluorescence was determined as described in FIGS. 3A-B. GFP fluorescence was normalized as percent GFP fluorescence, setting GFP following Ad/GFP_{tTA} at 100%. Averages and standard deviations of 3 experiments are shown.

FIG. 8—Schematic representation of vector utilizing full positive feedback loop with gene switch enhancement. TSP: tissue-specific promoter (e.g., AFP). TG: transgene (e.g., TNFa, TRAIL or FasL). In Tumor Cells: The tumor specific promoter is active, so some expression of tTA, LacR and the transgene is initiated. The LRE promoter is also active, so tTS is expressed, binds to TRE sequences, and downregulates LacR, tTA and the transgene. However, LacR in turn binds to the lacO sequences in LRE and suppresses tTS expression. Competition between the two TSi begins. Expression of tTA induces expression from promoters containing TRE, including its own. It competes with tTS for binding sites as well as increasing LacR expression. The result is that a positive feedback loop is established and more and more LacR, tTA and transgene are made, while tTS expression is more and more suppressed. In Non-Tumor Cells: The tumor specific promoter is not activated, but may have low or “leaky” expression close to background. Small amounts of tTA, LacR and transgene may be produced. LRE promoter is active, and tTS is expressed. It competes with the activity of LacR and tTA. However, not enough tTA is produced to initiate a positive feedback loop, and LacR levels are also too low to suppress tTS. tTS represses background expression from promoters containing TRE, with the result that virtually no tTA, LacR or transgene are produced in these cells.

### DESCRIPTION OF ILLUSTRATIVE EMBODIMENTS

When developing gene therapy vectors expressing toxic transgenes, several factors must be considered in the vector design. First, in order to prevent transgene-related systemic cytotoxicity, toxic gene expression must be restricted to only the cancer cell targets. Secondly, while the safety of the vector is important, highly induced expression in the target cells must not be compromised in the process since oftentimes, a major limitation of gene therapy vectors is insufficient gene expression to initiate a therapeutic effect. Conversely, highly induced transgene expression must also retain specificity in order to preserve the safety of the vector. Finally, it is important to consider the possibility that the vector's propagating cell line may also be susceptible to the effects of the toxic transgene. Therefore, it is necessary to also prevent transgene expression during the propagation and production of the gene therapy vector. For these reasons, it is important to consider regulation of transgene expression when designing gene therapy vectors.

I. The Present Invention

Strategies for restricting toxic gene expression both temporally and spatially include incorporating tissue or cancer-specific promoters and drug-inducible or repressible regulation systems (Kubo et al., 2003; Shirakawa et al., 2000; Rubinchik et al., 2001; Kistner et al., 1996; Kanai, 2001; Haviv and Curiel, 2001). Moreover, considerable progress has been made in achieving regulated gene expression through the use of chimeric (artificially constructed from modular domains of various endogenous and exogenous proteins) transcription factors responding to an externally supplied inducer drug (ligand). These transcriptional activation factors (TAFs) recognize cognate regulatory elements in the promoter of the target gene and the ligand regulates the interaction of the TAF with the DNA or the interaction of the DNA-bound factor with a transcriptional activation domain.

A simple and direct approach to combining cell type-specific expression pattern with drug-inducible expression system is to place the expression of TAF component(s) under the control of an appropriate cell-type specific promoter. One goal of such an embodiment of these systems would be to achieve full spatial, temporal and quantitative control over transgene expression, for the beneficial reasons outlined previously. Another, more limited goal would be an enhancement or amplification of transgene expression levels in specific target cells using the drug-mediated activation over the expression levels achievable when employing a cell type-specific promoter only. The latter goal would be desirable in the treatment of cancer using cytotoxic genes, where the major requirements are to limit transgene expression to tumor cells only, while generating as high expression levels as possible in those cells.

While a number of reports describes construction of such tissue specific/drug-inducible systems, the overall assessment of their performance is that at best it was mediocre, with deficiencies in either maximum achieved transgene expression levels (Smith-Arica et al., 2000), or in the length of time it took to initiate transgene expression (Burcin et al., 1999). These difficulties have been attributed to the reduced concentrations of TAFs in the cell, which itself is a consequence of using weaker tissue-specific promoters to drive TAF expression. In their standard embodiments, each of the aforementioned systems uses a powerful constitutively active promoter to drive the expression of its TAF component(s). Such a setup ensures high TAF concentrations in transduced cells and efficient generation of high transgene levels in the “on-state” of the system. Thus, there remains in the art a need for a mechanism that can generate high TAF expression levels in specific cell populations irrespective of the strength of the transcription activity of the chosen cell type-specific promoter, while still fully retaining the stringency of that promoter.

The regulatory system of the present invention is an improvement upon the currently developed tissue specific/drug-inducible expression systems that use cell type-specific promoters to drive the expression of drug-inducible TAFs. As discussed, usefulness of those systems is limited by the low activity of certain cell-type specific promoters. A first class of TS promoters are highly cell-type specific, but have low activity in target cells. This activity is frequently inadequate to achieve high TAF concentration inside the cell with the resulting degradation of system's performance. These limitations are overcome in the first variation of the present invention by incorporating both cell-type specific and TAF-responsive DNA elements into novel hybrid promoters to drive the expression of TAF genes.

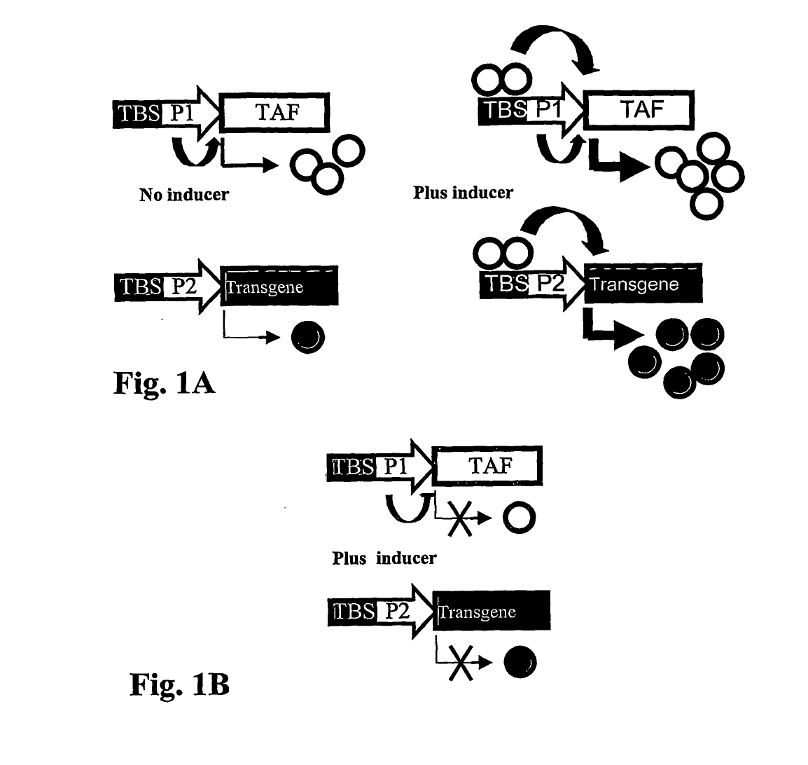

FIGS. 1A-B provide a schematic representation of one embodiment of this invention. Cognate TAF recognition elements are typically multiple repeats of a short sequence which form a TAF binding site (TBS) that is typically located in close proximity to a minimal promoter (P_{MIN}) driving the expression of the transgene. Such a general regulated promoter structure is shown in FIG. 1A. A hybrid cell type-specific/drug regulatable promoter can be constructed by inserting DNA elements with known cell type specific transcription regulation activity between the TBS and P_{MIN} sequences (FIG. 1A). Such a promoter can then be used to drive the expression of TAF. In a target cell, these elements will be active, initiating transcription of the TAF gene (1) and (2) production of TAF protein(s). In the absence of inducer, TAF would not bind to TBS and would not upregulate either its own expression or that of the transgene (3). If the inducer drug is present in the cell, TAF:drug complexes will form (4) and bind to TBS (5). This in turn will result in upregulation of transcription activity from the Pmin promoters driving both the transgene and the TAF gene (6). If the inducer concentration is sufficient to generate increasing concentrations of TAF:drug complexes, a positive feedback autoregulatory loop will be established driving the expression of TAF proteins. TAF expression will continue to increase until maximal positional and temporal occupancy of the TBS is achieved, at which time maximal transcriptional activity of both the hybrid promoter and the promoter driving the transgene will be reached (7). Since the activities of both hybrid and transgene promoters is dependent on the concentration of TAF:drug complexes, they will be regulatable by reducing or increasing concentrations of the inducer, which in turn will reduce or increase TAF:drug complex concentrations.

As indicated in FIG. 1B, this invention fully restricts expression system activity to target cells, since in the absence of cell type-specific element-mediated transcriptional activation, P_{MIN} promoter activity would be either naturally very low, or actually actively suppressed by those properties of cell type specific regulatory elements (i.e., binding sites for transcription suppressors) that insure their specificity (1). With such low promoter activity, levels of both, TAF and transgene will be subthershold (3), and not inducible even in the presence of the drug.

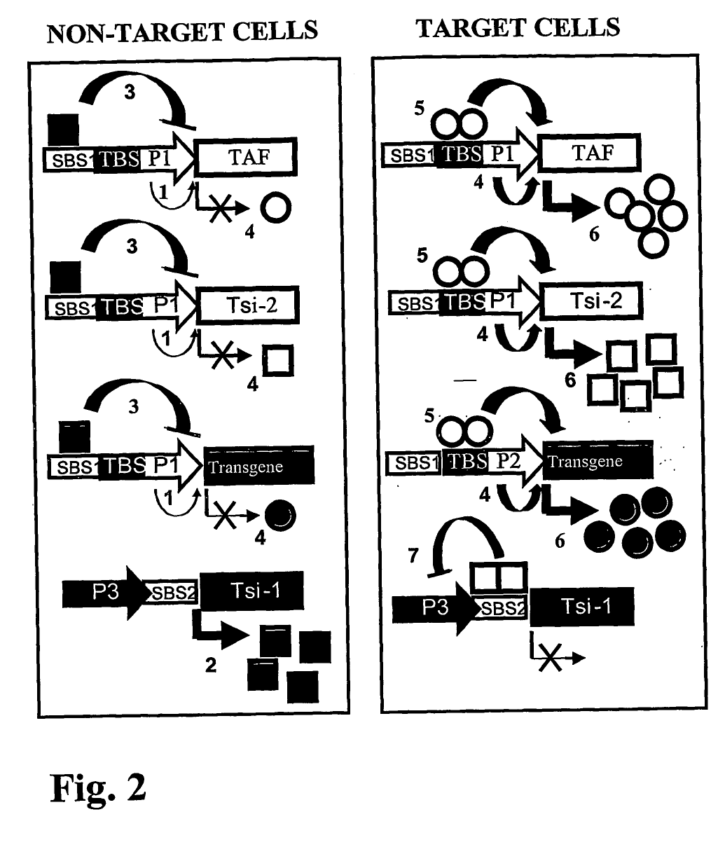

A second class of TS promoters has fairly high activity in target cells, but it is “leaky” having reduced but still substantial activity—in non-target cells. The second variation of the invention seeks to impose an “either/or” or “gene switch” expression pattern on the activity of these promoters, so that maximum expression can be reached in target cells, but all background activity in non-target cells is shut off. FIG. 2 is a schematic representation of a generalized version of this variation. This system utilizes two transcriptional suppressor genes (Tsi), TSi-1 and TSi-2, which have different DNA binding site specificities and may utilize either the same or two different mechanisms of transcriptional silencing. As in the first variation, the TAF gene is placed under the control of the hybrid promoter, while the transgene is driven either by the hybrid promoter or by a promoter only having the TAF responsive element (as shown). However, both promoters are modified to include a region containing multiple binding sites for TSi-1 (SBS). In addition, the same promoter that drives TAF is also used to drive the expression of TSi-2. TSi-1 expression is driven by a promoter that is constitutively active in all or a vast majority of cells, but which has been modified by the incorporation of an SBS region for TSi-2.

As shown in the left (non-target cell) panel of FIG. 2, upon introduction into a cell, the expression of TSi-1 initiates from its constitutive promoter (1). At the same time, TSREs of the “leaky” class 2 TS promoter will result in some expression of TAFs and TSi-2 proteins (2). As shown in FIG. 2, TSi-1 acts to suppress expression of transgene, TAF and TSi-2 genes (3), while TSi-2 acts to suppress TSi-1 expression (4). In non-target cells, where the activity of the constitutive promoter is significantly higher than that of the TSREs, TSi-1 activity will dominate, with the rapid establishment of essentially complete suppression of transgene, TAF and TSi-2 expression. In target cells (right panel, FIG. 2), TSi competition will also occur, but with a different outcome. Here, TSRE activity is higher than that of the constitutive promoter, so that more TAFs and TSi-2 proteins will be initially made (5). Once TAF levels are high enough, it will boost the activity (positive feedback loop) of its own expression (6), as well as increased expression of TSi-2 and the transgene (7). Higher TSi-2 levels are able to begin inhibiting TSi-1 expression (8), with the result that TSi-1 suppression of the other three promoters becomes weaker and weaker (9). Very quickly, TSi-1 expression is almost completely suppressed, and maximum levels of TAF, TSi-2 and transgene are reached (10). The schematic shown in FIG. 2 assumes that all transcription-regulating components (TAF, TSi-1 and TSi-2) are in an activated state, i.e., fully capable of binding to their cognate sites and performing their expression regulating functions. In principle, each of the three components can be regulated by a different small drug, so that highly complex and variable transgene expression patterns are possible.

In the present study, the inventors describe a specific version of the foregoing approach for inducing potent prostate-specific transgene expression incorporating elements of the Tet-off regulation system with the prostate-specific ARR2PB promoter. This regulation system demonstrates an enhancement of the transcriptional activity of the ARR2PB promoter without losing specificity. The positive feedback loop—prostate specific (PFLPS) regulation system has three characteristics that make it unique: (1) the newly developed TRE-ARR2PB promoter; (2) induction of a prostate-specific positive feedback loop; and (3) the cloning of the entire system into a single recombinant Ad vector, thus preventing the need for coinfection with two separate Ad vectors.

More specifically, the inventors combined TRE elements that respond to the Tet activator, with the prostate specific ARR2PB promoter. By combining these two elements in a hybrid regulatory region, and linking the hybrid region to the coding sequence for the Tet activator, the inventors were able to establish a positive feedback loop with prostate specificity (PFLPS), which generated high levels of prostate-specific expression of a marker polypeptide, GFP, driven from either a typical Tet-responsive promoter or from the TRE-ARR2PB hybrid promoter. Interestingly, activity from the PFLPS regulation system was at least 1.5-fold higher than the highly induced Tet-regulated system.

Normally one would expect that the consequence of such highly induced expression from a tissue-specific vector would be a loss in tissue-specificity. However, this was not the case. Even at MOI as high as 1000, the Ad/GFP_{PFLPS} vector demonstrated a retention in prostate-specificity. Notably, the Ad/GFP_{PFLPS} vector demonstrated little GFP expression in HepG2 cells at MOI 1000. This lack of transgene expression in liver-derived cells is significant since Ad vectors typically accumulate in the liver following systemic injection. Therefore, liver toxicity due to nonspecific transgene induction is less of an issue when the PFLPS system is utilized to control the expression of toxic transgenes.

Having demonstrated the feasibility of the PFLPS system, the inventors also envision other organ-restricted cancer gene therapy applications, using other transgenes. Candidate toxic transgenes for the gene therapy treatment of cancers include TRAIL (Rubinchik et al., 2003; Seol et al., 2003; Voelkel-Johnson et al., 2002), Bax (Andriani et al., 2001; Komatsu et al., 2000; Lee et al., 2000; Shinoura et al., 2000), and suicide genes such as herpes simplex virus thymidine kinase (HSV-tk) (Fillat et al., 2003; Nishihara et al., 1998; Yazawa et al., 2002; Kim et al., 2002). Additionally, conditionally-replicating adenovirus (CRAd) vectors have recently gained attention as a potential gene therapy vector for the treatment of cancers (van Beusechem et al., 2003; Yamamoto et al., 2003; Wildner, 2003). Incorporation of cancer-specific regulation of Ad early genes could further produce a potent yet safe vector for the treatment of cancers.

Moreover, the PFLPS regulation system can be used in non-cancer embodiments, as the positive feedback loop concept can be transferred to other tissue types by simply incorporating different tissue-specific promoters, thereby expanding its potential utility to include gene therapy of genetic disorders, development gene-based vaccines expressing immunogenic bacterial or viral antigens, and development of new animal models that require highly induced, organ-restricted expression of a particular gene of interest. Finally, the effective transcriptional regulation afforded by PFLPS could be combined with current methods of transductional regulation including manipulation (e.g., Ad fiber knob; Volk et al., 2003; Buskens et al., 2003; Belousova et al., 2002; Wesseling et al., 2001; Heideman et al., 2001; Vigne et al., 2003; Nakamura et al., 2003; use of bi-specific 13 antibodies; van Beusechem et al., 2003; Jongmans et al., 2003; Nettelbeck et al., 2004; Henning et al., 2002; Kashentseva et al., 2002) to further improve the targeting of gene therapy vectors to specific cell types and therefore increase the specificity and the safety of the vectors.

The present invention is exemplified by a single expression vector, an adenovirus, that provides two expression cassettes—one for the TAF protein, which is part of the positive feedback loop that results in high level TAF expression, and another for the selected transgene. While the inventors contemplate advantages to the use of a single expression vector, the present invention also contemplates separating these two transcription units into separate vectors. For example, an adenovirus (or other vector) comprising the TAF coding region placed under the control of the TRE/TS hybrid promoter may be provided without any further modifications. In such a case, there would be the need to generate a new vector using one engineering step—to introduce the cassette for the transgene of interest, linked to a TAF responsive promoter.

To summarize, the inventors have developed and characterized a novel transcriptional regulatory system that demonstrates highly induced, prostate-specific expression without a loss in specificity. Such a regulation system can be altered to include tissue or cancer-specific promoters in the place of the ARR2PB promoter, as well as any desired transgene, and thus is ideal for a variety of molecular genetic and gene therapeutic applications that require highly induced, organ-restricted expression of a particular gene of interest. Thus, the PFLPS regulation system serves as an exciting new strategy to deliver therapeutic genes for a multitude of molecular genetic and therapeutic applications.

II. Transcriptional Activating and Silencing Factors

In one aspect of the invention, the present invention relies on the use of transcriptional activating factors, and the genetic elements through which they act. In more particular embodiments, the present invention also utilizes transcriptional silencers to repress or limit transcription in non-target cells. Various systems are described below.

Experience with natural inducible promoters led to the formulation of a currently applied set of requirements for an “ideal” regulatable gene expression system for gene therapy. These include: (1) ligand-induced expression with dose-response and reproducible ON-OFF kinetics; (2) a subthreshold level of transgene expression in an uninduced (OFF) state; (3) an exogenous ligand (drug) that can be safely and repeatedly administered; (4) a transgene promoter with DNA elements not found elsewhere in the cell's genome and which are recognized by a modular transcriptional regulator (protein or a complex of proteins); (5) a transcriptional regulator (TAF) that binds to unique DNA sequences in the transgene promoter with high specificity and affinity, but only upon interaction with the ligand; (6) an interaction between the ligand and the TAF that is specific and exclusive and does not perturb any other activities or functions of the target tissue or of the host organism as a whole.

Several small molecule ligands have been employed to mediate regulated gene expressions, either in tissue culture cells and/or in transgenic animal models. The following systems are frequently used in current regulated gene expression applications. Overall, roughly similar performance parameters have been reported for each of these systems, with the choice between them depending largely on the nature of the application.

A. Transcriptional Activating Factors

1. Tetracycline-Inducible System

Tetracycline-inducible systems have been described in the literature by several groups. Gossen and Bujard, (1992); Gossen et al., (1995); Kistner et al., (1996). Chimeric tetracycline-repressed transactivator (tTA) was generated by fusing an activation domain from herpes simplex virus VP16 protein to the class E tetR protein (from Tn10 in E. coli). In the absence of tet, the tetR domain of tTA binds selectively and tightly to a synthetic DNA region called tetracycline response element (TRE), comprising seven repeats of tetO that were placed upstream of a minimal hCMV promoter. Once the TAF is bound near the promoter, its VP16 domain transactivates the target gene expression to very high levels. Binding of tet or other tet-like drugs such as doxycycline (dox) to tetR results in a conformational change and loss of tetR binding to the operator. A “mirror image” system was developed when tetR mutations conferring a reverse phenotype were isolated. In contrast to wild-type tetR, the reverse mutant requires dox to bind tetO and fails to do so in the drug's absence. The reverse tet transactivator (rtTA) activates gene expression in the presence of dox, rendering the system more suitable for therapeutic applications. Further improvements were made by replacing the VP16 domain of rtTA with better-tolerated and less immunogenic transcription activating peptides from NF-κB p65 protein.

Although this system performs very well in established cell lines and transgenic animals, its delivery to somatic cells using gene therapy vectors results in detectable basal expression levels. This promiscuity has been attributed to the inherent activity of the minimum hCMV promoter as well as to the presence of IFNα-stimulated response elements in the TRE. Although very low compared to the induced activity of this system, this basal expression may be sufficient to generate undesired toxicity in the case of especially potent cytotoxic agents. Recently, the system has been improved by incorporating tetracycline regulated transcription supressors to reduce background expression. Freundlieb et al., (1999). These proteins are fusions between tetR (class G) and the transcription inhibiting KREB domain of kid-1 protein. In the absence of the drug, they bind to TRE and suppress basal activity. When the drug is added, these regulators vacate the site, allowing rtTA to bind and activate transcription.

2. Mifepristone (RU486) Inducible System

Mifepristone (RU486) inducible system has been described in the literature. Wang, (1994); Wang et al., (1997). This system is based on the mutated progesterone nuclear receptor which has low affinity to progesterone and very high affinity to progestin antagonists such as mifepristone (MFP or RU486). The truncated ligand-binding domain of this mutant receptor was fused with the yeast GAL4 DNA-binding domain and the activation domain of VP16 or NF-κB p65 to generate the TAF for this system. The MFP-inducible promoter typically contains 4 or more copies of the GAL4 upstream activation sequence (UAS) fused to a minimal promoter (TATA box or TK promoter). The TAF binds to the GAL4 UAS of this promoter and induces target gene expression only when MFP is administered. Uniqueness of the UAS in mammalian cells and very low minimal promoter activity (below detection threshold) in the absence of activation combine to deliver exceptionally high stringency of this system. Animal studies have shown that full activation of the system requires MFP levels substantially lower than those needed to antagonize progesterone binding in humans.

3. Ecdysone-Inducible System

The ecdysone-inducible system has been described by No et al., (1996). The ecdysone system (ERS) is based on the insect hormone ecdysone and its functional receptor, EcR. Its TAF is assembled when a chimeric EcR derivative EcR/VP16 forms a heterodimer with retinoid X receptor (RXR□) in the presence of muristerone A, which is a synthetic analog of ecdysone. This hybrid receptor recognizes a modified DNA element consisting of ecdysone and glucocorticoid response element half-sites that is not naturally found in mammalian cells. Recently, improved ERS were developed that do not require RXRα overexpression but are able to utilize endogenously available RXRα levels, simplifying the use of this system in vivo. Although promising, the safety of ERS components have not been fully characterized in animal or human models. Administration of muristerone A or other ecdysone-like drugs may have some unforeseen effects in mammals, and it is not yet known whether the presence of pesticides with ecdysone-like structures in food and water will interfere with regulation.

4. Rapamycin-Inducible System

A rapamycin-inducible system has been described in the literature by Spencer et al., (1993) and Magari et al., (1997). This system is notable for using only human protein-derived components, which may give it an advantage in human gene therapy applications since it should exhibit low immunogenicity. Rapamycin, an antibiotic produced by Streptomyces hygroscopicus, binds to immunophilin proteins such as FK506-binding protein FKBP and thereby induces them to form complexes with the signaling proteins such as the lipid kinase homologue FRAP. The rapamycin-binding domain of FRAP was fused to the transcriptional activation domain from the p65 subunit of human NF-κB, while 3 copies of the rapamycin-binding domain of FKBP12 were fused to a novel DNA-binding domain called ZFHD1, which itself is a fusion of zinc fingers from egr-1 and oct-1.37 proteins and recognizes a unique synthetic DNA sequence. These fusion proteins are non-functional until they interact with rapamycin and heterodimerize. The ternary drug-protein complex then functions as a TAF, recognizing and binding to multiple copies of ZFHD1-binding element upstream of a minimal promoter driving the transgene expression. Rapamycin-regulated system has been reported to deliver low background and high induced levels of expression, demonstrating its potential. One disadvantage of this system is the requirement to use rapamycin at levels that are immunosuppressive. Currently, rapamycin derivatives with reduced immunosuppressive properties are being tested.

5. GAL4-VP16 System

Eukaryotic transcriptional regulatory proteins are typified by the Saccharomyces yeast GAL4 protein, which was one of the first eukaryotic transcriptional activators on which these functional elements were characterized. GAL4 is responsible for regulation of genes which are necessary for utilization of the six carbon sugar galactose. Galactose must be converted into glucose prior to catabolism; in Saccharomyces, this process typically involves four reactions which are catalysed by five different enzymes. Each enzyme is encoded by a GAL gene (GAL 1, 2, 5, 7, and 10) which is regulated by the transactivator GAL4 in response to the presence of galactose.

Each GAL gene has a cis-element within the promoter, termed the upstream activating sequence for galactose (UAS_{G}), which contains 17-basepair sequences to which GAL4 specifically binds. The GAL genes are repressed when galactose is absent, but are strongly and rapidly induced by the presence of galactose. GAL4 is prevented from activating transcription when galactose is absent by a regulatory protein GAL80. GAL80 binds directly to GAL4 and likely functions by preventing interaction between GAL4's activation domains and the general transcriptional initiation factors. When yeast are given galactose, transcription of the GAL genes is induced. Galactose causes a change in the interaction between GAL4 and GAL80 such that GAL4's activation domains become exposed to allow contact with the general transcription factors represented by TFIID and the RNA polymerase II holoenzyme and catalyse their assembly at the TATA-element which results in transcription of the GAL genes.

The functional regions of GAL4 have been carefully defined by a combination of biochemical and molecular genetic strategies. GAL4 binds as a dimer to its specific cis-element within the UAS_{G} of the GAL genes. The ability to form tight dimers and bind specifically to DNA is conferred by an N-terminal DNA-binding domain. This fragment of GAL4 (amino acids 1-147) can bind efficiently and specifically to DNA but cannot activate transcription. Two parts of the GAL4 protein are necessary for activation of transcription, called activating region 1 and activating region 2. The activating regions are thought to function by interacting with the general transcription factors. The large central portion of GAL4 between the two activating regions is required for inhibition of GAL4 in response to the presence of glucose. The C-terminal 30 amino acids of GAL4 bind the negative regulatory protein GAL80; deletion of this segment causes constitutive induction of GAL transcription. The VP16 fragment is a transactivation domain from the herpes simplex virus VP16 protein. A fusion product made from the DNA binding portion of GAL4 and VP16 creates a powerful transactivator of appropriate GAL4 promoters.

B. Transcriptional Silencers

1. Lac Repressor Regulated System

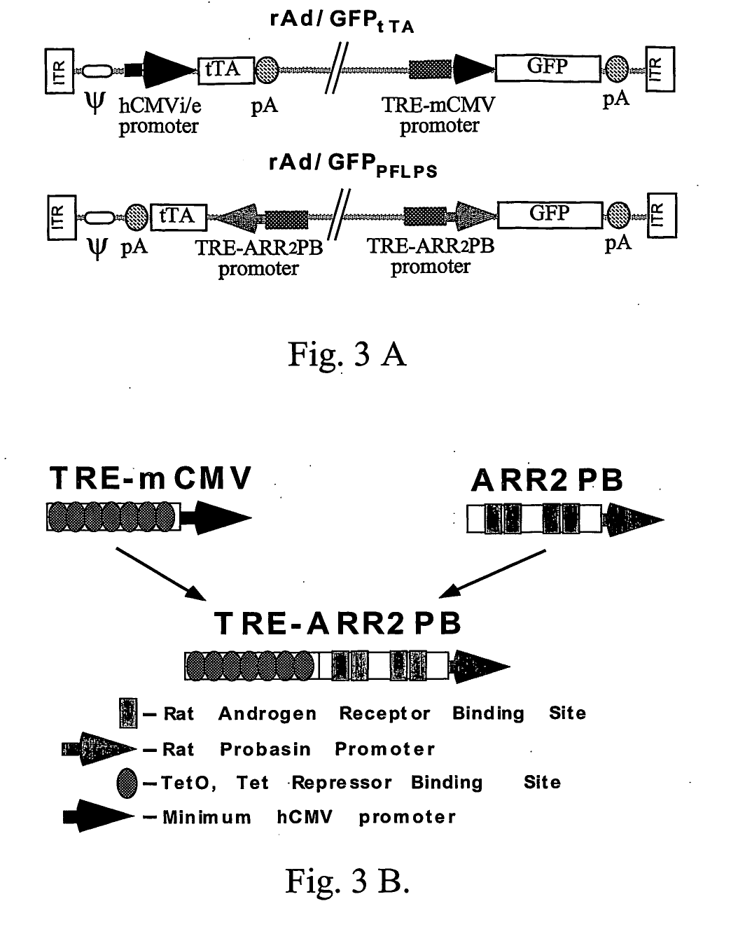

A lac repressor regulated system has been reported. Wyborski and Short, (1991), Fieck et al., (1992), Wyborski et al., (1996). In the bacterial lac operon system, the Lac repressor protein (LacR) is constitutively expressed and binds to its operator region, lacO, with very high affinity and specificity. When lactose is available, it binds to LacR, changing its conformation and releasing it from lacO, thereby allowing RNA polymerase binding to the promoter and transcription of the lactose metabolizing enzymes. Expression regulating systems that utilize LacR and lacO in eukaryotic cells have been developed, and are available commercially (e.g., LacSwitch from Stratagene). All of them utilize natural regulatory mechanism of the lac operon, with lacO sites placed near transcription initiation site, with the hope that LacR binding there will interfere with RNA Polymerase II interaction with the promoter. Typically, a lactose analog such as IPTG (isopropyl β-D-thiogalactopyranoside), is used to release LacR from the lacO sites, thus allowing some regulated transcription from the promoter. An efficient variant of this regulatory system was developed in our lab. It utilizes a synthetic LacR-responsive promoter (LRE), constructed by inserting two lacO operator sequences within the hCMV intermediate/early (hCMVie) promoter/enhancer, such that they flank the TATA box (see FIG. 3B). In this system, the LRE promoter behaves much like its parental hCMVie promoter, except that when LacR is expressed in the cell, it binds to the lacO sites of LRE and blocks RNA polymerase access to the TATA box, efficiently repressing transcriptional activity.

2. Tet and Other Bacterial/Phage Repressors

The tet repressor can be used in the similar manner to LacR, since it also binds tightly to its operator. TetRs from different bacterial strains have different operator sequences, so it would be possible to combine a TeR-based silencer with currently utilized tTA (a fusion of tetR and activating domain of VP16). In addition, other bacterial repressors can be used in the similar manner, for example cI of lambda phage. All of these unmodified repressors work by interfering with binding of TBP to TATA box, or with initiation of transcription, based on the positioning of their operator sites within the eukaryotic promoter. However, fusion proteins using DNA-binding domains of these repressors and a true eukaryotic transcriptional silencer (KREB domain of kid-1, used to make tTS hybrid silencer) can also be made.

III. Tissue Specific/Selective Promoters

In accordance with the present invention, tissue specific or selective promoters may be used in conjunction with the positive feedback loop expression system, described further below. The expression system relies, in the first instance, on the ability of a tissue specific promoter, when combined with TRE elements, to drive the expression of a transcriptional transactivator, which then acts to induce expression from a responsive promoter of interest. In fact, the promoter need not be entirely specific for a given cell or tissue but, rather, should be active preferentially or selective in a particular cell type, for example, a tumor cell. In other words, a small amount of expression in normal tissues, as compared to tumor tissues, may be tolerated. The following specific or preferential, promoters are specifically contemplated for use in accordance with the present invention.

A. Carcinoembryonic Antigen (CEA) Promoter

CEA is a membrane glycoprotein that is overexpressed in many carcinomas and is widely used as a clinical tumor marker. Paxton et al. (1987); Thompson et al. (1991). Sequence analysis has identified several molecules that are closely related to CEA, including non-specific cross-reacting antigens (NCA) and biliary glycoprotein. Neumaier et al. (1988); Oikawa et al. (1987); Hinoda et al. (1991). CEA is expressed at low levels in some normal tissues and is usually overexpressed in malignant colon cancers and other cancers of epithelial cell origin. Both CEA and NCA expression is fairly homogenous within metastatic tumors, presumably due to the important functional role of these antigens in metastasis. Robbins et al. (1993); Jessup and Thomas (1989).

The cis-acting sequence that confers expression of the CEA gene (SEQ ID NO:1) on certain cell types has been identified and analyzed. Hauck and Stanners (1995); Schrewe et al. (1990); Accession Nos. Z21818 and AH003050. It consists of approximately 400 nucleotides upstream from the translational start codon and has sequence homology with a similar sequence in NCA. Schrewe et al. (1990). This promoter has been used to drive some suicide genes and to mediate cell killing in tumor xenografts of stably transfected cells. Osaki et al. (1994); Richards et al. (1995). However, its application in gene therapy is limited by its relatively low transcriptional activity. To solve this problem, Kijima et al. recently used the Cre/loxP system to enhance transgene expression from the CEA promoter. Kijima et al. (1999). In their system, a stuffer DNA flanked by a loxP sequence was placed between a transgene and a strong upstream promoter. For coadministration with a second vector expressing a Cre gene driven by a CEA promoter, the stuffer DNA was removed to permit expression of the transgene from its upstream promoter. However, this approach requires rearrangement of vector molecules and is limited by the transcriptional activity of the upstream promoter which could be weak in some cell types.

B. hTERT Promoter

Recently, the human telomerase reverse transcriptase (hTERT) has been cloned by several groups and found to be expressed at high levels in primary tumors and cancer cell lines, but repressed in most somatic tissues. Nakamura et al. (1997); Meyerson et al. (1997); Kilian et al. (1997); Harrington et al. (1997). Data suggest that hTERT is a key determinant of telomerase activity. This includes the finding that hTERT expression is highly correlated with telomerase activity and that ectopic expression of hTERT in telomerase-negative cells is sufficient to reconstitute telomerase activity and extend the life span of normal human cells. Nakamura et al. (1997); Meyerson et al. (1997); Kilian et al. (1997); Harrington et al. (1997); Weinrich et al. (1997); Nakayama et al. (1998); Counter et al. (1998); Bodnar et al. (1998). More recently, it was reported that ectopic expression is required, but not sufficient, for direct tumorigenic conversion of normal human epithelial and fibroblast cells. Hahn et al. (1999).

The promoter region of the hTERT gene also has been cloned. Takakura et al. (1999); Horikawa et al. (1999); Cong et al. (1999); Accession Nos. AB016767 and AF097365. The promoter is high Gly/Cys-rich and lacks both TATA and CAAT boxes, but contains binding sites for several transcription factors, including Myc and Sp1. SEQ ID NO:3 and SEQ ID NO:5. Deletion analysis of the hTERT promoter identified a core promoter region of about 200 bp upstream of the transcription start site. Transient assays revealed that he core promoter is significantly activated in cancer cell lines but is repressed in normal primary cells.

C. PSA Promoter

Prostate specific antigen (PSA) or KLK3 as it is sometimes called, is a serine protease which is synthesized primarily by both normal prostate epithelium and the vast majority of prostate cancers. Accession No. S81389. The expression of PSA is mainly induced by androgens at the transcriptional level via the androgen receptor (AR). The AR modulates transcription through its interaction with its consensus DNA binding site, GGTACAnnnTGTT/CCT (SEQ ID NO:7), termed the androgen response element (ARE). Schuur et al. (1996). The core PSA promoter region exhibits low activity and specificity, but inclusion of the PSA enhancer sequence which contains a putative ARE increases expression, specifically in PSA-positive cells. Expression can be further increased when induced with androgens such as dihydrotestosterone, Latham et al. (2000).

D. AFP Promoter

Alpha-fetoprotein (AFP) is expressed at high levels in the yolk sac and fetal liver and at low levels in the fetal gut. Accession No. L34019. AFP transcription is dramatically repressed in the liver and gut at birth to levels that are barely detectable by postnatal day 28. This repression is reversible as the AFP gene can be reactivated during liver regeneration and in hepatocellular carcinomas. Previous studies in cultured cells and transgenic mice identified five distinct regions upstream of the AFP gene that control its expression. The promoter and three enhancers functioned as positive regulatory elements, whereas the repressor acted as a negative element. The promoter resides within the 250 bp directly adjacent to exon 1. The repressor, a 600 bp region located between −250 and −850, is required for postnatal AFP repression. Further upstream at −2.5, −5.0 and −6.5 kb are three enhancers termed Enhancer I (EI), EII, and EIII. These three enhancers are active, to varying degrees, in the three tissues where AFP is expressed.

E. Probasin and ARR2PB Promoters

One of the most well-characterized proteins uniquely produced by the prostate and regulated by promoter sequences responding to prostate-specific signals, is the rat probasin protein. Study of the probasin promoter region has identified tissue-specific transcriptional regulation sites, and has yielded a useful promoter sequence for tissue-specific gene expression. The probasin promoter sequence containing bases −426 to +28 of the 5′ untranslated region, has been extensively studied in CAT reporter gene assays (Rennie et al., 1993). Prostate-specific expression in transgenic mouse models using the probasin promoter has been reported (Greenberg et al., 1994). Gene expression levels in these models parallel the sexual maturation of the animals with 70-fold increased gene expression found at the time of puberty (2-6 weeks). The probasin promoter (−426 to +28) has been used to establish the prostate cancer transgenic mouse model that uses the fused probasin promoter-simian virus 40 large T antigen gene for targeted over expression in the prostate of stable transgenic lines (Greenberg et al., 1995). Thus, this region of the probasin promoter is incorporated into the 3′ LTR U3 region of the RCR vectors thereby providing a replication-competent MoMLV vector targeted by tissue-specific promoter elements.

The probasin promoter confers androgen selectivity over other steroid hormones, and transgenic animal studies have demonstrated that the probasin promoter will target androgen, but not glucocorticoid, regulation in a prostate-specific manner. Previous probasin promoters either targeted low levels of transgene expression or became too large to be conveniently used. Thus, a probasin promoter was designed that would be small, yet target high levels of prostate-specific transgene expression (Andriani et al., 2001). This promoter is ARR2PB which is a derivative of the rat prostate-specific probasin promoter which has been modified to contain two androgen response elements. ARR2PB promoter activity is tightly regulated and highly prostate specific and is responsive to androgens and glucocorticoids.

F. Other Tissue Specific/Preferential Promoters

Other tissue specific or preferential promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse α2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

Muscle specific promoters, and more particularly, cardiac specific promoters, also are known in the art. These include the myosin light chain-2 promoter (Franz et al., 1994; Kelly et al., 1995), the a actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the α7 integrin promoter (Ziober and Kramer, 1996), the brain natriuretic peptide promoter (Lapointe et al., 1995) and the α B-crystallin/small heat shock protein promoter (Gopal-Srivastava, 1995), a myosin heavy chain promoter (Yamauchi-Taklihara et al., 1989) and the ANF promoter (LaPointe et al., 1988).

IV. Therapeutic Transgenes

In accordance with the present invention, a selected gene or polypeptide may refer to any protein, polypeptide, or peptide. A therapeutic gene or polypeptide is a gene or polypeptide which can be administered to a subject for the purpose of treating or preventing a disease. For example, a therapeutic gene can be a gene administered to a subject for treatment or prevention of cancer. Examples of therapeutic genes include, but are not limited to, Rb, CFTR, p16, p21, p27, p57, p73, C-CAM, APC, CTS-1, zac1, scFV ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, Bax, Bak, Bik, Bim, Bid, Bad, Harakiri, Fas-L, mda-7, fus, interferon α, interferon β, interferon γ, ADP, p53, ABLI, BLC1, BLC6, CBFA1, CBL, CSFIR, ERBA, ERBB, ERBB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

Other examples of therapeutic genes include genes encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemiceflulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, β-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase.

Therapeutic genes also include genes encoding hormones. Examples include, but are not limited to, genes encoding growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

V. Expression Constructs and Gene Delivery

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1990 and Ausubel et al., 1996, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

In the present invention, particular embodiments will provide a promoter comprising one or more transcription response elements (TREs) that are activated by a transcription activation factor (TAF). These elements may be part of a natural promoter that regulated by the TAF, or they may be incorporated in to a synthetic promoter. In one embodiment, the invention contemplates a hybrid promoter that includes TREs in combination with elements from tissue specific promoters, such that transcription remains tissue specific, but is enhanced in the presence of TAF. Generally, the spacing between promoter elements frequently is flexible, so that promoter function is preserved even when elements like TREs are introduced near the tissue specific promoter elements. For example, in the tk promoter, the spacing between promoter elements can be increased to 50 bp apart before activity begins to decline. Depending on the promoter, it appears that individual elements can function either cooperatively or independently to activate transcription.

A promoter may be one naturally-associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. The promoter may be heterologous or endogenous.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picomavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

In order to propagate an expression vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, YFP, CFP, RFP, fluorescein, and rhodamine, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

A. Non-Viral Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for drug resistance and thus provides easy means for identifying transformed cells. The pBR plasmid, or other microbial plasmid, cosmid or phage would also contain, or be modified to contain, the expression cassettes of interest.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

B. Non-Viral Gene Transfer

Suitable methods for nucleic acid delivery for transformation of a cell for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

1. Ex Vivo Transformation

Methods for transfecting vascular cells and tissues removed from an organism in an ex vivo setting are known to those of skill in the art. For example, canine endothelial cells have been genetically altered by retrovial gene transfer in vitro and transplanted into a canine (Wilson et al., 1989). In another example, yucatan minipig endothelial cells were transfected by retrovirus in vitro and transplanted into an artery using a double-balloon catheter (Nabel et al., 1989). Thus, it is contemplated that cells or tissues may be removed and transfected ex vivo using the nucleic acids of the present invention. In particular aspects, the transplanted cells or tissues may be placed into an organism. In preferred facets, a nucleic acid is expressed in the transplanted cells or tissues.

2. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

3. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize. (U.S. Pat, No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 9217598, incorporated herein by reference. Other examples of species for which protoplast transformation has been described include barley (Lazzeri, 1995), sorghum (Battraw et al., 1991), maize (Bhattachajee et al., 1997), wheat (He et al., 1994) and tomato (Tsukada, 1989).

4. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

5. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

6. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

7. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

8. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

9. Microprojectile Bombardment

Microprojectile bombardment techniques can be used to introduce a nucleic acid into at least one, organelle, cell, tissue or organism (U.S. Pat. Nos. 5,550,318, 5,538,880, and 5,610,042, and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

C. Viral Vectors

The ability of certain viruses to infect cells via receptor-mediated endocytosis, and in some cases to integrate into host cell genome, has made them attractive candidates for gene transfer into host cells and expression of foreign genes. Although some viruses that can accept foreign genetic material are limited in the number of nucleotides they can accommodate, and may be limited in the range of cells they infect, viruses have been demonstrated to successfully effect gene expression, both in vitro and in vivo, making them ideally suited for rapid, efficient, heterologous gene expression. Techniques for preparing replication-defective and replication-competent viruses are well known in the art.

1. Adenovirus

Adenovirus is a non-enveloped double-stranded DNA virus. The virion consists of a DNA-protein core within a protein capsid. Virions bind to a specific cellular receptor, are endocytosed, and the genome is extruded from endosomes and transported to the nucleus. The genome is about 36 kB, encoding about 36 genes. In the nucleus, the “immediate early” E1A proteins are expressed initially, and these proteins induce expression of the “delayed early” proteins encoded by the E1B, E2, E3, and E4 transcription units. Virions assemble in the nucleus at about 1 day post infection (p.i.), and after 2-3 days the cell lyses and releases progeny virus. Cell lysis is mediated by the E3 11.6K protein, which has been renamed “adenovirus death protein” (ADP).

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

Adenovirus may be any of the 51 different known serotypes or subgroups A-F. Adenovirus type 5 of subgroup C is the human adenovirus about which the most biochemical and genetic information is known, and it has historically been used for most constructions employing adenovirus as a vector. Recombinant adenovirus often is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Viruses used in gene therapy may be either replication-competent or replication-deficient. Generation and propagation of the adenovirus vectors which are replication-deficient depends on a helper cell line, the prototype being 293 cells, prepared by transforming human embryonic kidney cells with Ad5 DNA fragments; this cell line constitutively expresses E1 proteins (Graham et al., 1977). However, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Adenovirus growth and manipulation is known to those of skill in the art, and exhibits broad host range in vitro and in vivo. This group of viruses can be obtained in high titers, e.g., 10⁹-10¹³ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Ad vectors are based on recombinant Ad's that are either replication-defective or replication-competent. Typical replication-defective Ad vectors lack the E1A and E1B genes (collectively known as E1) and contain in their place an expression cassette consisting of a promoter and pre-mRNA processing signals which drive expression of a foreign gene. These vectors are unable to replicate because they lack the E1A genes required to induce Ad gene expression and DNA replication. In addition, the E3 genes can be deleted because they are not essential for virus replication in cultured cells. It is recognized in the art that replication-defective Ad vectors have several characteristics that make them suboptimal for use in therapy. For example, production of replication-defective vectors requires that they be grown on a complementing cell line that provides the E1A proteins in trans.

Several groups have also proposed using replication-competent Ad vectors for therapeutic use. Replication-competent vectors retain Ad genes essential for replication, and thus do not require complementing cell lines to replicate. Replication-competent Ad vectors lyse cells as a natural part of the life cycle of the vector. An advantage of replication-competent Ad vectors occurs when the vector is engineered to encode and express a foreign protein. Such vectors would be expected to greatly amplify synthesis of the encoded protein in vivo as the vector replicates. For use as anti-cancer agents, replication-competent viral vectors would theoretically be advantageous in that they would replicate and spread throughout the tumor, not just in the initially infected cells as is the case with replication-defective vectors.

Yet another approach is to create viruses that are conditionally-replication competent. Onyx Pharmaceuticals recently reported on adenovirus-based anti-cancer vectors which are replication-deficient in non-neoplastic cells, but which exhibit a replication phenotype in neoplastic cells lacking functional p53 and/or retinoblastoma (pRB) tumor suppressor proteins (U.S. Pat. No. 5,677,178). This phenotype is reportedly accomplished by using recombinant adenoviruses containing a mutation in the E1B region that renders the encoded E1B-55K protein incapable of binding to p53 and/or a mutation(s) in the E1A region which make the encoded E1A protein (p289R or p243R) incapable of binding to pRB and/or p300 and/or p107. E1B-55K has at least two independent functions: it binds and inactivates the tumor suppressor protein p53, and it is required for efficient transport of Ad mRNA from the nucleus. Because these E1B and E1A viral proteins are involved in forcing cells into S-phase, which is required for replication of adenovirus DNA, and because the p53 and pRB proteins block cell cycle progression, the recombinant adenovirus vectors described by Onyx should replicate in cells defective in p53 and/or pRB, which is the case for many cancer cells, but not in cells with wild-type p53 and/or pRB.

Another replication-competent adenovirus vector has the gene for E1B-55K replaced with the herpes simplex virus thymidine kinase gene (Wilder et al., 1999a). The group that constructed this vector reported that the combination of the vector plus gancyclovir showed a therapeutic effect on a human colon cancer in a nude mouse model (Wilder et al., 1999b). However, this vector lacks the gene for ADP, and accordingly, the vector will lyse cells and spread from cell-to-cell less efficiently than an equivalent vector that expresses ADP.

The present inventor has taken advantage of the differential expression of telomerase in dividing cells to create novel adenovirus vectors which overexpress an adenovirus death protein and which are replication-competent in and, preferably, replication-restricted to cells expressing telomerase. Specific embodiments include disrupting E1A's ability to bind p300 and/or members of the Rb family members. Others include Ad vectors lacking expression of at least one E3 protein selected from the group consisting of 6.7K, gp19K, R1Da (also known as 10.4K); RIDβ (also known as 14.5K) and 14.7K. Because wild-type E3 proteins inhibit immune-mediated inflammation and/or apoptosis of Ad-infected cells, a recombinant adenovirus lacking one or more of these E3 proteins may stimulate infiltration of inflammatory and immune cells into a tumor treated with the adenovirus and that this host immune response will aid in destruction of the tumor as well as tumors that have metastasized. A mutation in the E3 region would impair its wild-type function, making the viral-infected cell susceptible to attack by the host's immune system. These viruses are described in detail in U.S. Pat. No. 6,627,190.

Other adenoviral vectors are described in U.S. Pat. Nos. 5,670,488; 5,747,869; 5,981,225; 6,069,134; 6,136,594; 6,143,290; 6,410,010; and 6,511,184.

2. AAV Vectors

Adeno-associated virus (AAV) is an attractive vector system for use in the cell transduction of the present invention as it has a high frequency of integration and/or it can infect nondividing cells, thus making it useful for delivery of genes into cells, for example, in tissue culture (Muzyczka, 1992) and/or in vivo. AAV has a broad host range for infectivity (Tratschin et al., 1984; Laughlin et al., 1986; Lebkowski et al., 1988; McLaughlin et al., 1988). Details concerning the generation and/or use of rAAV vectors are described in U.S. Pat. Nos. 5,139,941 and 4,797,368, each incorporated herein by reference.

Studies demonstrating the use of AAV in gene delivery include LaFace et al. (1988); Zhou et al. (1993); Flotte et al. (1993); and/or Walsh et al. (1994). Recombinant AAV vectors have been used successfully for in vitro and/or in vivo transduction of marker genes (Kaplitt et al., 1994; Lebkowski et al., 1988; Samulski et al., 1989; Yoder et al., 1994; Zhou et al., 1994; Hermonat and/or Muzyczka, 1984; Tratschin et al., 1985; McLaughlin et al., 1988) and genes involved in diseases (Flotte et al., 1992; Luo et al., 1996; Ohi et al., 1990; Walsh et al., 1994; Wei et al., 1994). Recently, an AAV vector has been approved for phase I trials for the treatment of cystic fibrosis.

AAV is a dependent parvovirus in that it requires coinfection with another virus (either adenovirus and/or a member of the herpes virus family) to undergo a productive infection in cultured cells (Muzyczka, 1992). In the absence of coinfection with helper virus, the wild type AAV genome integrates through its ends into chromosome 19 where it resides in a latent state as a provirus (Kotin et al., 1990; Samulski et al., 1991). rAAV, however, is not restricted to chromosome 19 for integration unless the AAV Rep protein is also expressed (Shelling and/or Smith, 1994). When a cell carrying an AAV provirus is superinfected with a helper virus, the AAV genome is “rescued” from the chromosome and/or from a recombinant plasmid, and/or a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and/or an expression plasmid containing the wild-type AAV coding sequences without the terminal repeats, for example pIM45 (McCarty et al., 1991; incorporated herein by reference). The cells are also infected and/or transfected with adenovirus and/or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions and/or cell lines containing the AAV coding regions and/or some and/or all of the adenovirus helper genes could be used (Yang et al., 1994; Clark et al., 1995). Cell lines carrying the rAAV DNA as an integrated provirus can also be used (Flotte et al., 1995).

3. Retroviral Vectors

Retroviruses have promise as gene delivery vectors due to their ability to integrate their genes into the host genome, transferring a large amount of foreign genetic material, infecting a broad spectrum of species and/or cell types and/or of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and/or directs synthesis of viral proteins. The integration results in the retention of the viral gene sequences in the recipient cell and/or its descendants. The retroviral genome contains three genes, gag, pol, and/or env that code for capsid proteins, polymerase enzyme, and/or envelope components, respectively. A sequence found upstream from the gag gene contains a signal for packaging of the genome into virions. Two long terminal repeat (LTR) sequences are present at the 5′ and/or 3′ ends of the viral genome. These contain strong promoter and/or enhancer sequences and/or are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and/or env genes but without the LTR or packaging components is constructed (Mann et al., 1983). When a recombinant plasmid containing a cDNA, together with the retroviral LTR and/or packaging sequences is introduced into this cell line (by calcium phosphate precipitation for example), the packaging sequence allows the RNA transcript of the recombinant plasmid to be packaged into viral particles, which are then secreted into the culture media (Nicolas and/or Rubenstein, 1988; Temin, 1986; Mann et al., 1983). The media containing the recombinant retroviruses is then collected, optionally concentrated, and/or used for gene transfer. Retroviral vectors are able to infect a broad variety of cell types. However, integration and/or stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to and infected cells bearing the erythropoietin (EPO) receptor. This was achieved by inserting a portion of the EPO sequence into an envelope protein to create a chimeric protein with a new binding specificity.

4. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and/or herpes simplex virus may be employed. They offer several attractive features for various cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and/or reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and/or pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and/or organization of the genome. Because HSV is large, incorporation of multiple genes and/or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and/or can be grown to high titers. Thus, delivery is less of a problem, both in terms of volumes needed to attain sufficient MOI and in a lessened need for repeat dosings.

5. Modified Viruses

In still further embodiments of the present invention, the nucleic acids to be delivered are housed within a virus that has been modified or engineered to express a specific binding ligand or otherwise alter its tissue specificity. The virus particle will thus bind to the cognate receptors of the target cell and deliver its contents to the cell. An approach designed to allow specific targeting of retrovirus vectors has been recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and/or class II antigens, they demonstrated the infection of a variety of cells that bore those surface antigens with an ecotropic virus in vitro (Roux et al., 1989).

The present invention contemplates manipulation of the Ad fiber knob (Volk et al., 2003; Buskens et al., 2003; Belousova et al., 2002; Wesseling et al., 2001; Heideman et al., 2001; Vigne et al., 2003; Nakamura et al., 2003) and use of bi-specific 13 antibodies (van Beusechem et al., 2003; Jongmans et al., 2003; Nettelbeck et al., 2004; Henning et al., 2002; Kashentseva et al., 2002) to modify Ad host range.

VI. Cancer Therapies

In the context of the present invention, it is contemplated that the vectors of the present invention may be used to deliver therapeutic genes to an individual to treat cancer. Cancers contemplated by the present invention include, but are not limited to, breast cancer, lung cancer, head and neck cancer, bladder cancer, bone cancer, bone marrow cancer, brain cancer, colon cancer, esophageal cancer, gastrointestinal cancer, gum cancer, kidney cancer, liver cancer, nasopharynx cancer, ovarian cancer, prostate cancer, skin cancer, stomach cancer, testis cancer, tongue cancer, or uterine cancer. In particular embodiments, treatment of prostate cancer is contemplated. The following genes are exemplary of those that may be used with vectors according to the present invention.

The vectors of the present invention may be delivered orally, nasally, intramuscularly, intraperitoneally, or intratumorally. In some embodiments, local or regional delivery of vectors according to the present invention, alone or in combination with an additional therapeutic agent, to a patient with cancer or pre-cancer conditions will be a very efficient method of delivery to counteract the clinical disease. Similarly, chemo or radiotherapy may be directed to a particular, affected region of the subject's body. Regional chemotherapy typically involves targeting anticancer agents to the region of the body where the cancer cells or tumor are located. Other examples of delivery of the compounds of the present invention that may be employed include intraarterial, intracavity, intravesical, intrathecal, and intrapleural routes.

Intraarterial administration is achieved using a catheter that is inserted into an artery to an organ or to an extremity. Typically, a pump is attached to the catheter. Intracavity administration describes drugs that are introduced directly into a body cavity such as intravesical (into the bladder), peritoneal (abdominal) cavity, or pleural (chest) cavity. Agents can be given directly via catheter. Intravesical chemotherapy involves a urinary catheter to provide drugs to the bladder, and is thus useful for the treatment of bladder cancer. Intrapleural administration is accomplished using large and small chest catheters, while a Tenkhoff catheter (a catheter specially designed for removing or adding large amounts of fluid from or into the peritoneum) or a catheter with an implanted port is used for intraperitoneal chemotherapy. Abdomen cancer may be treated this way. Because most drugs do not penetrate the blood/brain barrier, intrathecal chemotherapy is used to reach cancer cells in the central nervous system. To do this, drugs are administered directly into the cerebrospinal fluid. This method is useful to treat leukemia or cancers that have spread to the spinal cord or brain.

Alternatively, systemic delivery of the agents may be appropriate in certain circumstances, for example, where extensive metastasis has occurred. Intravenous therapy can be implemented in a number of ways, such as by peripheral access or through a vascular access device (VAD). A VAD is a device that includes a catheter, which is placed into a large vein in the arm, chest, or neck. It can be used to administer several drugs simultaneously, for long-term treatment, for continuous infusion, and for drugs that are vesicants, which may produce serious injury to skin or muscle. Various types of vascular access devices are available.

A. Inhibitors of Cellular Proliferation

Tumor suppressors function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p₂₁^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, mda-7, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fms, trk, ret, gsp, hst, abl, E1A, p300, genes involved in angiogenesis (e.g., VEGF, FGF, thrombospondin, BAI-1, GDAIF, or their receptors) and MCC.

B. Regulators of Programmed Cell Death

Apoptosis, or programmed cell death, is an essential process in cancer therapy Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists. Members of the Bcl-2 that function to promote cell death include Bax, Bak, Bik, Bim, Bid, Bad and Harakiri.

C. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that antisense mRNA or siRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

D. Toxins

In the context of this application, toxins are cytotoxic proteins that are able to damage and kill cells through direct effect of their function. These agents may be derived from non-human sources, such as diptheria and botulin toxins from bacteria, or the cytosine deaminase from yeast, or thymidine kinase from herpes simplex virus, or fusogenic envelope proteins from various human and non-human viruses (for example, VSVG and GLV). Alternatively, they may be natural human proteins, such as the extracellular inducers of apotosis from the TNF family, including (but not limited to) TNFα, FasL, TRAIL, and apoptin, or perforin and granzyme B that are employed by the cytolytic T-lymphocytes to kill target cells.

1. Fas-L

Fas Ligand (CD95L or APO-1L) is a 40 kDa type II membrane protein belonging to the Tumor Necrosis Factor (TNF) family. Its receptor, Fas (CD95 or APO-1) is a 45 kDa type I membrane protein belonging to the TNF/NGF (Nerve Growth Factor) superfamily of receptors (Suda and Nagata, 1994; Takahashi 1994). Following engagement with its ligand, Fas functions to initiate an apoptotic signal in Fas-bearing cells. This signal originates at the death inducing signaling complex (DISC), which forms just below the cell's surface on the cytoplasmic domain of Fas. The DISC, in part, is composed of Fas, an adapter molecule (FADD/MORT), and pro-caspase 8 (FLICE/MACH) (Ashkenazi and Dixit, 1998). Upon Fas stimulation, FADD and pro-caspase 8 are recruited to Fas enabling pro-caspase 8 to autocatalytically activate itself (Medema, 1997). Active caspase 8, in turn, cleaves and/or activates several downstream substrates including the effector caspases 3 and 7 (Muzio, 1997). These effector caspases are responsible for cleaving vital cellular substrates (for example, RB, PARP, and lamins), which ultimately leads to apoptosis.

Fas is a widely expressed protein found on the plasma membrane in most tissues including prostate. In contrast, FasL expression appears to be more tightly regulated on the plasma membrane. Membrane FasL (mFasL) expression has only been detected in immune privileged tissues such as testis (Bellgrau, 1995), retina (Griffith, 1995), cornea (Stuart, 1997), and in immunological cells (T and NK cells) (Rouvier, 1993; Arase, 1995; Stalder, 1994). However, several recent reports suggest that mFasL occurs in both normal and malignant prostate, although this data remains controversial. Liu et al. (1998) detected mFasL expression on the surface of cultured LNCaP cells using FACS analysis. In the same report, they also detected soluble FasL (sFasL) in the culture media of PC-3, DU145, LNCaP cells, and within the intraluminal secretions of normal prostate epithelial cells (human). Soluble FasL is generated by matrix metalloproteinase (MMP) cleavage of membrane bound FasL (mFasL) between a.a. 127 and 128 (Mariani, 1995; Kayagaki, 1995; Powell, 1999). In contrast to the aforementioned report, Sasaki et al. (1998) was unable to detect mFasL expression in 21 of 21 localized PCa specimens using a similar approach. Cleavage of mFasL by the MMP may explain these discrepancies.

Despite the inconsistencies regarding surface FasL expression in prostate, several experiments have demonstrated that a functional Fas-mediated apoptotic pathway exists in the prostate. This evidence comes both from in vitro and in vivo studies. In vitro, some PCa cell lines (PPC-1, ALVA-31, JCA-1 (Hedlund, 1998); PC-3 (Rokhlin, 1997)) are sensitive to Fas-mediated apoptosis when challenged with a Fas agonist, i.e., anti-Fas antibody or FasL expressing effector cells (Hyer, 2000; Rokhlin, 1997; Hedlund, 1998). Other PCa cell lines (DU145, ND1, JCA-1 (Rokhlin, 1997), PC-3 (Frost, 1997; Uslu, 1997) were found to be resistant when challenged with a Fas agonist. This resistance, however, was overcome by pretreatment using sub-toxic concentrations of cyclohexamide, cis-diamminedichloroplatinum(II) (CDDP), VP-16, adriamycin (ADR), or camptothecin (Rhokhlin, 1997; Frost, 1997; Uslu, 1997; Costa-Pereira, 1999). These chemotherapeutic drugs have different mechanisms of action, but presumably function to remove a block in the Fas-mediated pathway and allow the death signal to proceed. Interestingly, LNCaP cells were found to remain Fas-resistant even after drug pre-treatment. However, Hyer et al. (2000) demonstrated that LNCaP cells were uniformly sensitive to Fas-mediated apoptosis following treatment with a FasL expressing adenovirus. There is also in vivo evidence suggesting a functional Fas-mediated apoptotic pathway present in both rat and mouse prostate models.

One of the limitations in PCa gene therapy is delivery of the therapeutic gene to every cell in the tumor. FasL gene therapy attempts to address this problem by taking advantage of the “bystander effect.” The bystander effect occurs when the number of apoptotic cells is greater than the number of cells expressing the transgene. Potentially, this can allow for complete regression of a solid tumor without having to deliver FasL to every cell. FasL can initiate the bystander effect three different ways: (1) by remaining associated with the FasL expressing cell; (2) by being released as a soluble form from the FasL expressing cell (Liu et al., 1998; Mariani, 1995); or (3) by being released as a membrane bound form in microvesicles (Martinez-Lorenzo, 1999). It has been shown in vitro that FasL expressing effector cells (K562-FasL cells) can kill the following Fas+ PCa cell lines: ALVA-31, TSU-PR1, PPC-1, and JCA-1 (Hedlund, 1999). In addition, Liu et al. (1998) has demonstrated that FasL derived from the media of cultured LNCaP cells was capable of inducing apoptosis in Fas+ Ramos cells (Liu, 1998). It is unclear in the above two studies whether the target cells were dying from sFasL or membrane bound FasL. The role sFasL plays in Fas-mediated apoptosis is controversial. Some reports suggest sFasL stimulates the Fas pathway (Liu et al., 1998), while others contend sFasL inhibits the pathway (Tanaka, 1998). In PCa, an in vivo bystander effect has not been demonstrated.

Using FasL to induce apoptosis in PCa is a promising new strategy. Recently it has been shown in vitro, that following transduction with a FasL expressing adenovirus, apoptosis occurs in the following PCa cell lines: LNCaP, PPC-1, TSU-Pr1, DU145, PC-3, JCA-1, and ALVA-31 (Hyer, 2000; Hedlund, 1999). Interestingly, adenovirus-mediated FasL delivery was capable of overcoming Fas-resistance in all cell lines determined to be resistant to antibodies with Fas agonistic characteristics. The mechanism whereby virally-expressed FasL overcomes Fas-resistance has not been determined (Hyer, 2000). Adenovirus-mediated FasL delivery has successfully been used to both reduce tumor burden and increased survival in the following human and mouse (in vivo) tumor models: glioma (Ambar, 1999), leiomyosarcoma (Aoki, 2000), colon carcinoma (Arai, 1997), and mouse renal carcinoma (Arai, 1997). Evidence suggests that observed tumor reduction is the result of the following two phenomena: 1) FasL induced apoptosis of Fas bearing cells, and 2) a FasL stimulated immune response. In the colon carcinoma model, elimination of the tumor was mediated exclusively by inflammatory cells (Arai, 1997). FasL expression has also been shown to be a potent chemoattractant for human neutrophils (Ottonello, 1999). However, it is still unclear exactly what role the immune system plays in eliminating FasL expressing tumor cells. With regard to PCa, Hedlund et al. (1999) has demonstrated that TSU-Pr1 cells, which were pre-infected with a FasL containing adenovirus and then subcutaneously injected into nude mice, exhibited reduced tumor potential compared to controls (Hedlund, 1999). However, further studies are necessary to determine the fill therapeutic value of FasL as an in vivo PCa gene therapy.

2. TRAIL

Another member of the TNF family is TNF-related apoptosis inducing ligand (TRAIL, Apo-2). Full length TRAIL is a 32 kDa protein, identified in 1995 as a novel membrane protein with amino acid similarity to TNF (23%) and FasL (28%) (Wiley, 1995). Like other members of the TNF family, TRAIL can induce receptor-mediated apoptosis by activating the caspase cascade (Kim, 2000). In contrast to FasL, which can cause severe hepatotoxicity and TNF which has been associated with septic shock, TRAIL can induce apoptosis in tumorigenic and transformed cells without adversely affecting normal cells. Safety studies in mice (Walczak, 1999) and cynomolgus monkeys (Ashkenzai, 1999) indicate that TRAIL is well tolerated in vivo, although some concern has been raised about its toxicity against primary cultures of human hepatocytes (Jo, 2000). The apparent lack of toxicity, coupled with the ability to kill a variety of tumor cells in vitro (Kim, 2000; Griffith, 1998) and in vivo (Walczak, 1999; Ashkenzai, 1999; Gliniak, 1999), has sparked great interest in the potential use of TRAIL as a novel anticancer agent. Although numerous tumor cell lines have been analyzed for susceptibility, receptor status, and mechanism of TRAIL-induced apoptosis, data obtained from prostate cancer cell lines is limited and sometimes contradictory.

Unlike other members of the TNF family, TRAIL is expressed in a wide variety of tissues (Wiley, 1995), and it was originally thought that susceptibility to TRAIL may be regulated by restrictive expression of a TRAIL receptor. To date, four ubiquitously expressed TRAIL receptors have been identified which raises the question of how normal tissues maintain resistance to TRAIL. The prostate is one of the tissues which express high levels of TRAIL (Wiley, 1995), and transcripts for each TRAIL receptor can be detected by RT-PCR in primary cultures of prostate epithelial cells (PrEC). Several hypotheses have been developed to explain the mechanism of resistance to TRAIL. TRAIL responses are mediated by a complex receptor system. Of the four TRAIL receptors that have been identified, two DR4/TRAIL-R1 (Pan, 1997) and DR5/TRAIL-R2 (Macfarlane, 1997; Bodmer, 2000) have functional death domains that can bind FADD or FADD-like adaptor molecules thereby initiating the caspase cascade and apoptosis (Kuang, 2000). The two remaining receptors either lack (DcR1/TRAIL-R3) (Pan, 1997; Macfarlane, 1997; Degliesposti, 1997) or have a truncated death domain (DcR2/TRAIL-R4) (Pan, 1998; Degliesposti, 1997) and are presumed to be decoy receptors. This was based on the observation that many tumor cells lack these receptors and over-expression in TRAIL-sensitive cells resulted in protection from TRAIL-induced apoptosis (Pan, 1998; Degliesposti, 1997). Subsequent studies examining numerous cell lines for TRAIL receptor expression, were unable to support this hypothesis because levels of receptors correlated poorly with TRAIL susceptibility (Griffith and Reed, 1998; Griffith and Lynch, 1998; Leverkus, 2000; Mitsiades, 2000). A second hypothesis, based on the observation that DcR2 can activate NFκB, suggested that decoy receptors may transduce anti-apoptotic signals (Degliesposti, 1997; Jeremias and Debatin; Jeremias et al., 1998). A later study found that DR4 and DR5 can also induce NFκB activation without any protective effects (Schneider, 1997; Chaudhary, 2000; Yamanaka, 2000). It has also been suggested that resistance to TRAIL may be determined by levels of intracellular inhibitors such as FLIP (Kim, 2000; Zhang, 1999). It is likely that a combination of TRAIL receptor levels, competing apoptotic and anti-apoptotic signals as well as intracellular levels of various pro- and anti-apoptotic proteins ultimately determine a cell's fate in response to TRAIL.

PrEC have been demonstrated to be resistant to TRAIL by several investigators (Ashkenazi, 1999; Griffith, 2000), (unpublished observations from our laboratory). In another study, the prostate adenocarcinoma cell lines PC3, Du145, and LNCaP were also found to be resistant to TRAIL-induced apoptosis and resistance did not correlate with TRAIL receptor levels as measured by RT-PCR (van Ophoven, 1999). Unfortunately, data regarding TRAIL susceptibility of PC3 and Du145 prostate cancer cell lines differ between reports. PC3 cells have lower levels of DcR1 and DcR2 transcript levels relative to Du145 and LNCap, and are still sensitive to TRAIL (Griffith, 2000; Yu, 2000). Over-expression of DR4 enhances this susceptibility (Yu, 2000). Yu et al. (2000) also observed Du145 to be sensitive to TRAIL-induced apoptosis which is not in agreement with observations made by Sun et al. (2000).

TRAIL resistant cells can be sensitized by inhibitors of RNA and protein synthesis (actinomycin D, cycloheximide) (Griffith, 1998; Thomas, 1998), chemotherapeutic agents (cisplatinum, etoposide, doxorubicin) (Kim, 2000; Ashkenazi, 1999; Gliniak, 1999; Keane, 1999; Gibson, 2000; Nagane, 2000) or radiation (Chinnaiyan, 2000). Yu et al. (2000) who found Du145 and PC3 cells susceptible to TRAIL-induced apoptosis, were unable to further enhance killing by co-treatment with cycloheximide (Yu, 2000). However, in studies in which these cells were found to be resistant to TRAIL, low concentrations of actinomycin D have been shown to convert Du145, LNCaP and PC3 cells to a TRAIL-sensitive phenotype (van Ophoven, 1999; Bonavida, 1999), indicating that the presence of intracellular inhibitors of apoptosis may mediate resistance. The synthetic retinoid CD437 also acts synergistically with TRAIL by upregulating DR5 (Sun, 2000).

The first in vivo studies in mice bearing mammary or colon cancer xenografts demonstrated that TRAIL administration significantly prolonged survival (Walczak, 1999; Ashkenazi, 1999; Gliniak, 1999). Furthermore, combination of TRAIL and the camptothecin, CPT-11, resulted in a high proportion of complete tumor regression in TRAIL sensitive tumors and dramatically slowed growth of TRAIL resistant tumors. One of the problems with in vivo use of TRAIL is the high concentration requirement, in part, because soluble TRAIL has a short half-life in plasma (about 32 minutes) (Ashkenazi, 1999) and an elimination half-life of less than 5 hours (Walczak, 1999). To improve delivery and better target TRAIL to the tumor site, Griffith et al. (2000) developed a TRAIL expressing adenoviral vector. Upon viral infection and production of TRAIL, sensitive targets such as PC3 cells were killed rapidly, whereas resistant targets such as PrEC were unaffected. Interestingly, PrEC still expressed adenovirally derived TRAIL and were able to kill PC3 cells in co-incubation experiments (Griffith, 2000). This suggests that not all tumor cells would have to be infected by the adenovirus as normal cells surrounding the tumor could aid in tumor cell apoptosis, i.e., a bystander effect.

3. TNF-α

Tumor Necrosis Factor-α (TNF-α), also known as cachectin, causes tumor necrosis in vivo. TNF-α is a 26 kDa membrane bound protein which is cleaved by TNF-α converting enzyme (TACE) to release the soluble 17 kDa monomer which forms homotrimers in circulation. Recombinant TNF-α is found as a homodimer, -trimer or ipentamer. TNF-α is expressed in many types of cells, primarily in macrophage cells, in response to immunological challenges such as bacteria (lipopolysaccharides), viruses, parasites, mitogens and other cytokines. As such, it plays roles in antitumor activity, immune modulation, inflammation, anorexia, cachexia, septic shock, viral replication and ematopoiesis. TNF-α causes cytolysis or cytostasis of many transformed cells, being synergistic with γ-interferon in its cytotoxicity. Although it has little effect on most cultured normal human cells, TNF-α is directly toxic to vascular endothelial cells.

E. Combination Therapy

Additional therapeutic agents contemplated for use in combination with a gene delivered using the vectors of the present invention. Traditional anticancer agents may include, but are not limited to, radiotherapy, chemotherapy, gene therapy, hormonal therapy or immunotherapy that targets cancer/tumor cells.

To kill cells, induce cell-cycle arrest, inhibit cell growth, inhibit metastasis, or otherwise reverse or reduce the malignant phenotype of cancer cells, using the methods and compositions of the present invention, one would generally contact a cell with a vector, liposome or viral particle according to the present invention in combination with an additional therapeutic agent. These treatments would be provided in a combined amount effective to inhibit cell growth and/or induce apoptosis in the cell. This process may involve contacting the cells with a vector, liposome or viral particle according to the present invention thereof in combination with an additional therapeutic agent or treatment at the same time. This may be achieved by contacting the cell with a single composition or pharmacological formulation that includes both agents, or by contacting the cell with two distinct compositions or formulations, at the same time, wherein one composition includes a vector, liposome or viral particle according to the present invention and the other includes the additional agent.

Alternatively, treatment with a vector, liposome or viral particle according to the present invention may precede or follow the additional agent treatment by intervals ranging from minutes to weeks. In embodiments where the additional agent is applied separately to the cell, one would generally ensure that a significant period of time did not expire between the time of each delivery, such that the agent would still be able to exert an advantageously combined effect on the cell. In such instances, it is contemplated that one would contact the cell with both modalities within about 12-24 hr of each other and, more preferably, within about 6-12 hr of each other, with a delay time of only about 12 hr being most preferred. Thus, therapeutic levels of the drugs will be maintained. In some situations, it may be desirable to extend the time period for treatment significantly (for example, to reduce toxicity). Thus, several days (2, 3, 4, 5, 6 or 7) to several weeks (1, 2, 3, 4, 5, 6, 7 or 8) may lapse between the respective administrations.

It also is conceivable that more than one administration of either a vector, liposome or viral particle according to the present invention in combination with an additional anticancer agent will be desired. Various combinations may be employed, where a vector, liposome or viral particle according to the present invention is “A” and the additional therapeutic agent is “B”, as exemplified below:
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B
   Other combinations are contemplated. Again, to achieve cell killing by the induction of apoptosis, both agents are be delivered to a cell in a combined amount effective to kill the cell.

1. Chemotherapeutic Agents

The present invention also contemplates the use of chemotherapeutic agents in combination with a vector, liposome or viral particle according to the present invention in the treatment of cancer. Examples of such chemotherapeutic agents may include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, gemcitabien, navelbine, farnesyl-protein transferase inhibitors, transplatinum, 5-fluorouracil and methotrexate, or any analog or derivative variant of the foregoing.

2. Radiotherapeutic Agents

Radiotherapeutic agents may also be use in combination with a vector, liposome or viral particle according to the present invention in treating a cancer. Such factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapeutic Agents

Immunotherapeutics may also be employed in the present invention in combination with a vector, liposome or viral particle according to the present invention in treating cancer. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

4. Surgery

It is further contemplated that a surgical procedure may be employed in the present invention. Approximately 60% of persons with cancer will undergo surgery of some type, which includes preventative, diagnostic or staging, curative and palliative surgery. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments maybe of varying dosages as well.

5. Hormonal Therapy

Hormonal therapy may also be used in conjunction with the vectors according to the present invention, or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. Increased intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

VII. Other Therapeutic Applications

In accordance with the present invention, it is contemplated that the methods and compositions disclosed herein can also be used in a variety of non-cancer related therapeutic applications. It is contemplated, for example, that any particular disorder, medical condition, or disease that can be treated or prevented by introducing a particular gene of interest into a cell can be treated or prevented by the present invention. Non limiting examples of such diseases include cystic fibrosis, AIDS, sickle cell anemia, adenosine deaminase deficiency, hemophilia, Gaucher's disease, diabetes, heart diseases, inflammatory diseases (e.g., rheumatoid arthritis, multiple sclerosis, inflammatory bowel disease, allergic asthma, etc.), manic depressive illnesses, and restenosis. The particular therapeutic gene for a given disease or condition can easily be identified by a person of ordinary skill in the art.

The method and composition of the present invention may also be used to treat or prevent neurodegenerative diseases by promoting neuronal regeneration processes. This can be done, for example, by stimulating the production of neuronal cell growth factors or cytokines. In particular embodiments, the selected polynucleotide may be a neurotrophic factor. A non-limiting example is a nucleotide that encodes neurotrophic factor (CNTF), brain-derived neurotrophic factor (BDNF), or glial cell line-derived neurotrophic factor (GDNF) (Mitsumoto et al., 1994; Gash et al., 1998, both herein incorporated by reference). Alternatively, the selected polynucleotide of the expression construct may optionally encode tyrosine hydroxylase, GTP cyclohydrolase 1, or aromatic L-amino acid decarboxylase (Kang, 1998, herein incorporated by reference). In still another embodiment, the therapeutic expression construct may express a growth factor such as insulin-like growth factor-1 (IGF-1) (Webster, 1997, incorporated herein by reference).

VIII. Pharmaceutical Formulations

Where clinical applications are contemplated, it will be necessary to prepare pharmaceutical compositions vectors, or any additional therapeutic agent disclosed herein in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes but is not limited to, oral, nasal or buccal routes. Alternatively, administration may be by orthotopic, intradermal, subcutaneous, intramuscular, intraperitoneal or intravenous injection. Such compositions would normally be administered as pharmaceutically acceptable compositions, described supra. The drugs and agents also may be administered parenterally or intraperitoneally. The term “parenteral” is generally used to refer to drugs given intravenously, intramuscularly, or subcutaneously.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation of vector constructs, liposome or virion particles include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent(s) of the present invention is determined based on the intended goal, for example (i) inhibition of tumor cell proliferation or (ii) elimination of tumor cells. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

### IX. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell Lines.

HEK293 (human embryonic kidney), LNCaP (human prostate cancer) and 22RV1 (mouse prostate cancer) cells were obtained from American Type Culture Collection (ATCC; Manassas, Va.). U343MG and U251MG (brain tumor) cell lines were obtained from the Brain Tumor Research Center Tissue Bank (Dept. of Neurological Surgery, UCSF, San Francisco, Calif.). All cell lines were maintained in media supplemented with 10% cosmic calf serum (CCS; HyClone, Logan, Utah), with HEK293 being maintained in DMEM, LNCaP and 22RV1 being maintained in RPMI, and U343MG and U251MG being maintained in MEM.

Construction of Plasmid Vectors.

The pUHD10-3 (containing the TRE promoter) and pUHD15-1 (containing the tTA gene) were generously provided by Hermann Bujard (Center for Molecular Biology, University of Heidelberg, Heidelberg, Germany). The ARR2PB (0.45 kb) promoter was developed in the laboratory of Robert J. Matusik (Department of Cell Biology, Vanderbilt University Medical Center, Nashville, Tenn.), who contributed the pARR2PB.PolI.TRZ-SK vector. ARR2PB is based on the minimal probasin promoter with a duplicated probasin androgen response region (ARR) upstream of it (Kasper et al., 1999). Construction of pLAd-CMV, pLAd-mcs and pRAd-T.GFP vectors has been described previously (Rubinchik et al., 2000). The inventors excised the ARR2PB promoter from pARR2PB.PolI.TRZ-SK and the tTA gene from pUHD15-1 and cloned them into pLAd-mcs to generate pLAd-2Pb.tTA (Rubinchik et al., 2001). The inventors excised the ARR2PB.tTA cassette from pLAd-2Pb.tTA and cloned it back in but in reverse to generate pLAd(2Pb.tTA)r. The inventors excised the TRE promoter from pUHD10-3 and cloned it upstream of the ARR2PB promoter in the pLAd(2Pb.tTA)r construct to generate pLAd(T2Pb.tTA.S)r (see FIG. 2A). The inventors cloned the TREARR2PB. tTA cassette in reverse orientation near the left ITR so that its promoter is away from the E1a enhancer region since we had previously found that the basal activity of both the TRE promoter and the ARR2PB promoter were significantly affected by interference from the E1a enhancer (Rubinchik et al., 2001). To construct the pRAd2T2Pb.GFP.B plasmid (see FIG. 2A), the inventors excised the TRE-ARR2PB promoter from pLAd-2Pb.tTA and cloned it in the place of the TRE promoter in pRAd2T.GFP.B (a plasmid closely related to pRAd-T.GFP).

Construction of Recombinant Adenoviral Vectors.

Construction of Ad/C.LacZ and Ad/GFP_{TET} has been described previously (Rubinchik et al., 2000). pLAd(T2Pb.tTA.S)ᵣ and pRAd²T²Pb.GFP.B plasmids were digested with Swa I and Spe I and ligated to an Ad5 genome backbone (Ad5sub360SR) digested on both ends with Xba I. The assembly of the Ad/GFP_{PFLPS} vector genome was constructed as described previously (Rubinchik et al., 2002; Rubinchik et al., 2002). All Ad vectors were based on Ad5sub360SR, which contains deletions in E3 and all E4 ORFs with the exception of ORF6.

Propagation and Titering of Recombinant Adenovirus Vectors.

All vectors were propagated in HEK293 cells, using standard procedures (Rubinchik et al., 2002; Rubinchik et al., 2002; Rubinchik et al., 2000). Briefly, HEK293 cells, which provide Ads E1a and E1b functions in trans, were transfected with the ligation mixture containing the recombinant adenovirus (rAd) vector DNA using Fugene 6 transfection reagent (Roche, Indianapolis, Ind.) and manufacturer's instructions. Transfected cells were maintained until adenovirus-related cytopathic effects (CPE) were observed (typically 7-14 days post-transfection), at which point the cells were collected. Vector propagation and amplification was then achieved by standard techniques. Briefly, adenoviral lysates from twenty-four 150 mm2 plates were banded twice on CsCl gradients and desalted twice with a PD-10 size exclusion column (Amersham Scientific, Piscataway, N.J.) into HEPES buffered saline (HBS; 21 mM HEPES, 140 mM NaCl, 5 mM KCl, 0.75 mM Na₂HPO₄.2H₂O, and 0.1% (w/v) dextrose; adjust pH with NaOH to 7.5; and filter sterilize) containing 5% glycerol, and stored at −70° C. All vectors were titrated on HEK293 16 cells infected in serial dilution on triplicate columns of 96-well plates for either GFP fluorescence or X-gal staining. GFP fluorescence was monitored with Axiovert-25 fluorescent microscope (Carl Zeiss, Germany) and FITC excitation/emission filter set (Chroma Technology Corp, Rockingham, Vt.) two days post-infection. Cells infected with Ad/C.LacZ were fixed two days post-infection with fixative solution (2% formaldehyde, 0.05% glutaraldehyde in 1× PBS) for 5 min at room temperature and then stained overnight at 37° C. in X-gal solution (1 mg/ml X-gal [5-Bromo-4-chloro-3-indolyl-βD-galactopyranoside], 5 mM potassium ferricyanide, 5 mM potassium ferrocyanide, 2 mM MgCl2 in 1× PBS). The resulting titers were scored as infectious units (IU) per ml.

Transfections and Infections In Vitro.

For plasmid DNA transfections, 1.0-2.5×10⁵ cells/well were seeded in 24-well plates and transfected 18 hr post-seeding using SuperFect reagent (Qiagen, Hilden, Germany) according to manufacturer's instructions. Cotransfection with pUHD 15-1 and pRAd²T.GFP.B (designated as Tet in FIG. 3) served as a positive control for GFP expression. pLAd-CMV served as an empty vector control for transfections. Co-transfection of pRAd²2Pb.GFP and pLAd-CMV served as a control for prostate-specific GFP expression (designated as ARR2PB in FIG. 3). For Ad vector infections, 1×10⁴ cells/well were seeded in 96-well plates or 1×10⁵ cells/well were seeded in 24-well plates. Seeded cells were infected 3 hours post-seeding at multiplicities of infection (MOI) of 0, 10, 50, 100, or 1000. MOI calculations were based on cell numbers at the time of seeding and on Ad vector titers based on IU/ml.

Quantification of GFP Expression.

In the transfection or infection studies, GFP fluorescence in cells was visualized 72 or 48 hours post-transduction, respectively, using Axiovert-25 fluorescent microscope with FITC filter set. For quantitative analysis of GFP activity, cells were lysed with 0.5% Triton x-100 in 1× PBS. Cell lysates were transferred to 96-well black microtiter plates (3MG Labtechnologies, Offenburg, Germany) and relative GFP fluorescence was measured using FLUOstar™ dual fluorescence/absorbance plate reader (BMG Labtechnologies) at excitation 485 nm and emission 520 nm.

RT-PCR.

Cells were seeded on 100 mm² plates. When cells reached 80-90% confluency, cells were trypsinized and counted the day of infection. Cells were resuspended in 10 ml serum-containing medium, infected at MOI 10, and plated onto 100 mm² plates. Two days post-infection, media was aspirated and cells were harvested in 1 ml TRI reagent (Sigma-Aldrich, St. Louis, Mo.). RNA was purified according to manufacturer's instructions. cDNA was synthesized from 1 μg RNA/sample using the RETROscript™ kit (Ambion Inc., Austin, Texas) according to manufacturer's instructions. Following reverse transcription, cDNA were amplified for either GFP or β-actin using GoTaq DNA Polymerase (Promega, Madison, Wis.) according to manufacturer's instructions. PCR was performed on the cDNA using the following sense and anti-sense primers: 5′-GCAAGGGCGAGGAGCTGTTCA-3′ (SEQ ID NO:8) and 5′-AAGTTCACCTTGATGCCGTTCTTC-3′ (SEQ ID NO:9) for GFP and 5′-GTGGGGCGCCCCAGGCACCA-3′ (SEQ ID NO:10) and 5′-CTCCTTAATGTCACGCACGATTTC-3′ (SEQ ID NO:11) for β-actin. PCR products were amplified by the following touchdown PCR program: 96° C. for 2 min; 12 cycles of 96° C. for 20 sec, 75° C. decreasing 1.5° C./cycle for 20 sec; 72° C. for 1 min; 13 cycles of 96° C. for 20 sec, 58° C. for 20 sec, 72° C. for 1 min; 72° C. for 10 min; hold at 4° C. PCR products were resolved on 1:1 mixture of 3% Synergel agarose clarifier additive (Diversified Biotech, Boston, Mass.) and 0.8% agarose (EM Science; Gibbstown, N.J.) in 1× TAE buffer.

### Example 2

### Tissue Specific Expression

The goal was to construct a complex adenovirus-based (rAd) vector capable of generating high expression levels of a pro-apoptotic FasL protein in prostate-derived cells but not in the cells of other origins. Previous studies indicated that high expression of FasL in prostate cancer cells could be achieved using a rAd vector delivering that gene under the control of tet-inducible system, and that this high expression was effective in eliciting apoptosis in those cells. Hyer et al., (2000). At the same time, the inventors were interested in increasing the safety of our therapy by transcriptionally restricting FasL expression to prostate cancer cells by using a synthetic promoter (ARR2PB) based on rat probasin promoter elements (FIG. 2A). Zhang et al., (2000). The levels of FasL expression achieved with ARR2PB were significantly lower that those generated by the tet inducible system, with the corresponding decrease in the levels of apoptosis. Rubinchik et al., (2001).

To achieve both high levels of expression and tight prostate cancer cell specificity, the inventors constructed a hybrid promoter by introducing the TRE upstream of the androgen response region of the ARR2PB (FIG. 3A). In the new vector, the tet transactivator (tTA) gene was placed under the control of this promoter in order to establish an autoregulatory positive feedback expression loop in androgen receptor-containing prostate cancer cells (FIG. 3B). The expression of the transgene (GFP in the case of the expression regulation experiments presented here) was also placed under the control of the hybrid promoter (FIG. 3B), with the result that significant level of transgene expression occurs in prostate-derived cells in the “OFF” state of the tet system. This was done for the following reasons: first, the TRE promoter of the tet-inducible system generates detectable background expression activity when used in rAd vectors, thus downgrading the cell-type specific expression pattern; second, the primary requirements of this embodiment are tight prostate cell specificity and high expression levels, and not the ability to regulate transgene expression by altering concentrations of tetracycline. The new rAd vector incorporating both of these expression cassettes was named rAd/GFP_{PFLPS}, for Positive Feedback Loop Prostate Specific (FIGS. 3A-B).

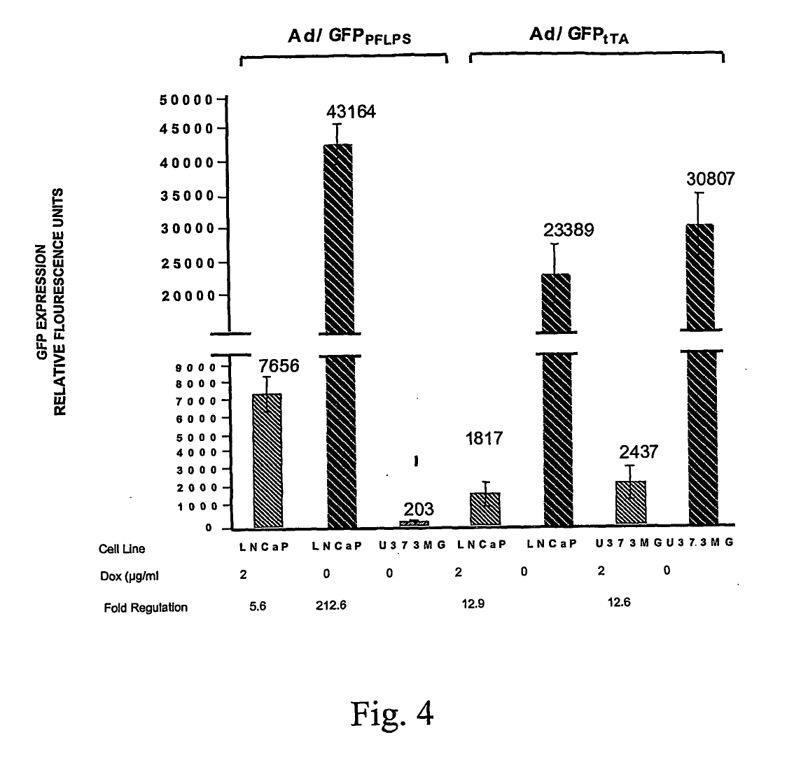

Basic parameters of the activity of the rAd/GFP_{PFLPS} vector are demonstrated in FIG. 4. In prostate cancer cell line LNCaP, high levels of GFP expression are generated following transduction with this vector. This activity decreases approximately 6-fold when doxycycline is added at levels sufficient to suppress tTA binding to TRE. The remaining activity is predominantly the result of the ARR2PB component function. In comparison, rAd vector delivering unmodified Tet-OFF system (FIG. 3B) generates lower GFP expression levels in LNCaP cells both in the presence and in the absence of dox. This vector generates essentially the same activity profile in non-prostate U373MG cell line, but GFP expression in rAd/GFP_{PFLPS}-transduced U373MG cells is virtually undetectable (FIG. 4).

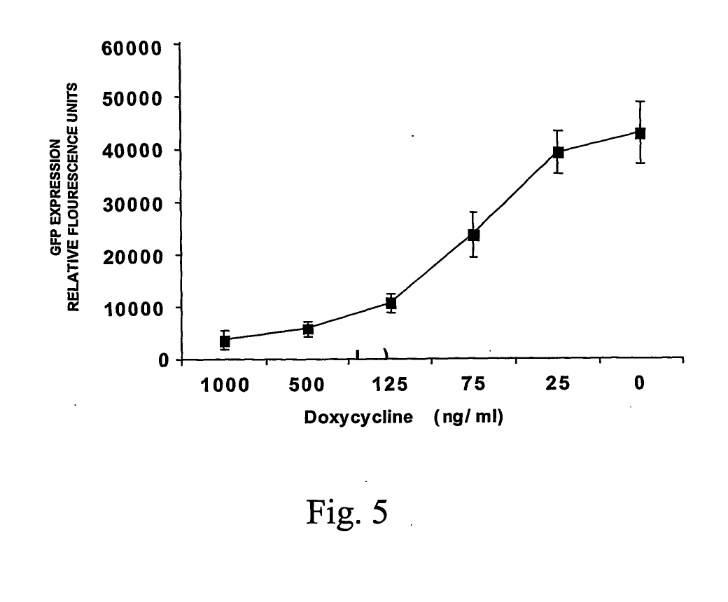

Although this embodiment is not specifically intended for regulated transgene expression, this aspect of the invention can nevertheless be convincingly demonstrated. FIG. 5 shows that GFP expression in LNCaP cells transduced with rAd/GFP_{PFLPS} vector can be regulated by changing the concentration of doxycycline in culture media.

### Example 3

### Tissue Specific Expression with Non-Target Suppression

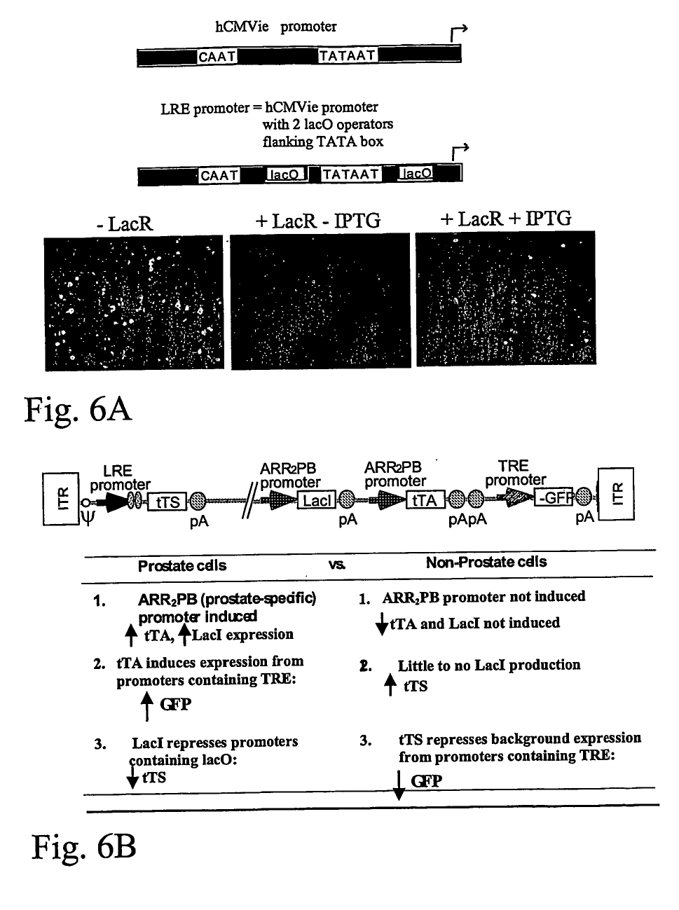

Another embodiment is based on a variation of the invention, which utilizes two transcriptional silencers in addition to TAF to regulate transgene expression. In this case, the goal was to evaluate the performance of the cross-inhibiting TSi proteins, and therefore the positive feedback loop portion of the strategy was not used. The system is again incorporated into a single complex Ad vector, with ARR2PB promoter driving both the expression of the tTA (TAF) and of the LacR (TSi-2). The transgene (GFP) is controlled by the tTA-inducible TRE promoter, while LacR-suppressible LRE promoter (FIG. 6A) drives the expression of the tTS (TSi-1). In this case, tetO sites in the TRE serve as binding sites for both tTA and tTS, acting as TBS and SBS-1 regions simultaneously. The new Ad vector incorporating all of these elements was named rAd/GFP_{PSTRGS}, for prostate-specific tet-regulated gene switch system (FIG. 6B).

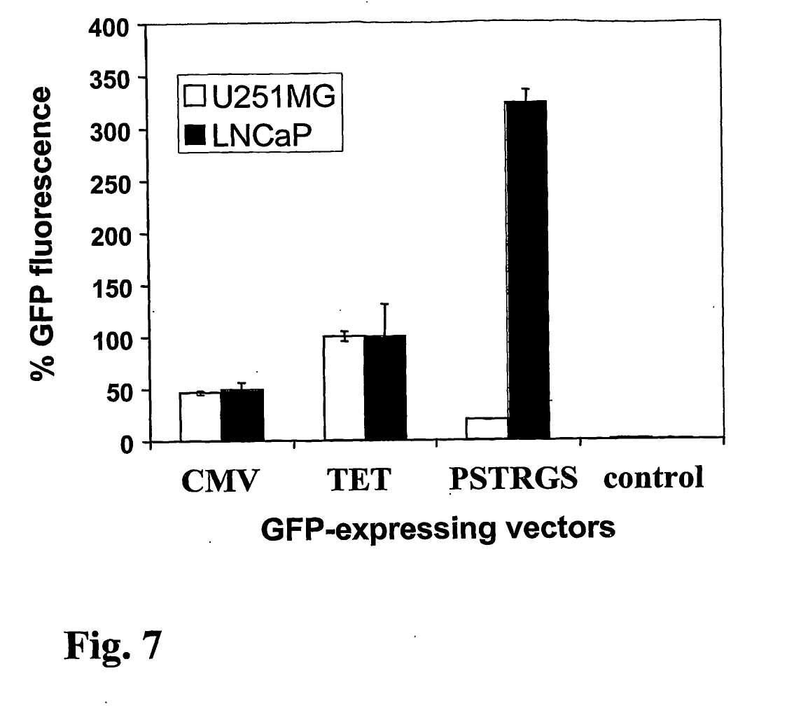

An example of the vector activity is shown in FIG. 7. The vector is highly efficient in prostate tumor cells, LNCaP, generating more activity than the controls. In non-prostate U251MG cells, vector-delivered GFP expression is greatly reduced, although some background remains. This experiment demonstrates the concept of switching between high and low expression levels based on the outcome of the competition between two cross-inhibiting transcriptional silencer.

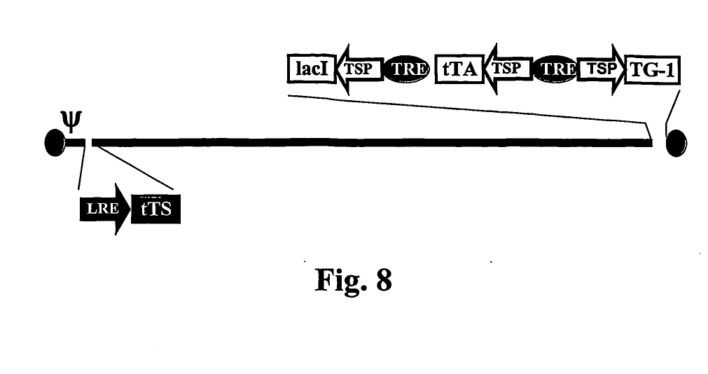

Complete integration of the genetic switch and the positive feedback loop components into a single system are expected to provide significant improvements in performance. A schematic version of one such vector, utilizing the elements already introduced in the descriptions of the PFLPS and PSTRGS-based vectors (FIGS. 3A-B and 6A-B), is shown in FIG. 8.

### Example 4

### Conditionally Replicating AD5 Vector

Another embodiment is based on the PFLPS system and was developed as an additional strategy for treatment of prostate cancer. The goal was to construct a conditionally-replicating adenovirus vector whose ability to propagate was specifically and tightly restricted to tumor cells of prostate origin. Previous variants of such vectors have been made, with replication specificity derived from the regulation of the activity of the adenovirus early gene, E1A, E1B or E4. However, it is known in the art that even very minor levels of activity of these early regulating genes are sufficient to generate some vector replication and propagation, so that these vectors could be described as semi-specific, with some level of viral replication and propagation in non-specific (non-prostate tumor) cells, typically at levels 2 to 3 orders of magnitude less than in target cells. Even these background levels may be problematic for the next generation of vectors, carrying potent cytotoxic and immunomodulating genes.

Tighter regulation can be achieved by controlling the expression of one of the late, or structural, proteins, since these are required in high amounts for effective capsid assembly. However, activity of current prostate-specific promoters is insufficient to provide required levels of these proteins. Therefore, the PFLPS will be used to generate high levels of one of the adenovirus late proteins, in this embodiment the fiber protein, in prostate tumor cells. Other Ad proteins may include, but are not limited to, the hexon, the penton, the 100K, the peptidase, the pre-terminal protein, the DNA polymerase, the DNA binding protein, and the 52K/55K protein.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

### REFERENCES

The following references, to the extent that they provide exemplary procedural or other details supplementary to those set forth herein, are specifically incorporated herein by reference:
U.S. Pat. No. 4,684,611
U.S. Pat. No. 4,797,368
U.S. Pat. No. 4,952,500
U.S. Pat. No. 5,139,941
U.S. Pat. No. 5,302,523
U.S. Pat. No. 5,322,783
U.S. Pat. No. 5,384,253
U.S. Pat. No. 5,464,758
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,670,488
U.S. Pat. No. 5,677,178
U.S. Pat. No. 5,677,178
U.S. Pat. No. 5,702,932
U.S. Pat. No. 5,736,524
U.S. Pat. No. 5,747,869
U.S. Pat. No. 5,780,448
U.S. Pat. No. 5,789,215
U.S. Pat. No. 5,925,565
U.S. Pat. No. 5,935,819
U.S. Pat. No. 5,945,100
U.S. Pat. No. 5,981,225
U.S. Pat. No. 5,981,274
U.S. Pat. No. 5,994,624
U.S. Pat. No. 6,069,134
U.S. Pat. No. 6,136,594
U.S. Pat. No. 6,143,290
U.S. Pat. No. 6,410,010
U.S. Pat. No. 6,511,184
U.S. Pat. No. 6,627,190
Almendro et al., J. Immunol., 157(12):5411-5421, 1996.
Ambar et al., Hum. Gene Ther., 10: 1641-1648, 1999.
Andriani et al., J. Natl. Cancer Inst., 93:1314-1324, 2001.
Aoki et al., Mol. Ther., 1:555-565, 2000.
Arai et al., Proc. Natl. Acad. Sci. USA, 94:13862-13867, 1997.
Arap et al., Cancer Res., 55(6):1351-1354, 1995.
Arase et al., J. Exp. Med., 181:1235-1238, 1995.
Ashkenazi and Dixit, Science, 281:1305-1308, 1998.
Ashkenazi et al., Clin. Invest., 104:155-162, 1999.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Bakhshi et al., Cell, 41(3):899-906, 1985.
Barnes et al., J. Biol. Chem., 272(17):11510-11517, 1997.
Bates, Mol. Biotechnol., 2(2):135-145, 1994.
Battraw and Hall, Theor. App. Genet., 82(2):161-168, 1991.
Bellagrau et al., Nature, 377:630-632, 1995.
Belousova et al., J. Virol., 76:8621-8631, 2002.
Bhattachajee et al., J. Plant Bioch. Biotech., 6(2):69-73. 1997.
Bhavsar et al., Genomics, 35(1):11-23, 1996.
Bodmer, Nature Cell Biol., 2:241-243, 2000.
Bodnar et al., Science, 279:349-352, 1998.
Bonavida et al., Intl. J. Oncology, 15:793-802, 1999.
Bower et al., J. Plant, 2:409-416. 1992.
Buising and Benbow, Mol. Gen. Genet., 243(1):71-81, 1994.
Burcin et al., Proc. Natl. Acad. Sci. USA, 96: 335-360, 1999.
Buskens et al., Ann. Surg., 238:815-824, 2003.
Caldas et al., Nat. Genet., 8(1):27-32, 1994.
Cancer Facts & Figures, American Cancer Society, 2003.
Carbonelli et al., FEMS Microbiol. Lett., 177(1):75-82, 1999.
Chandler et al., Proc. Natl. Acad. Sci. USA, 94(8):3596-601, 1997.
Chang et al., Hepatology, 14:124A, 1991.
Chaudhary et al., Oncogene, 19:4451-4460, 2000.
Chen and Okayama, Mol. Cell Biol., 7(8):2745-2752, 1987.
Cheng et al., Cancer Res., 54(21):5547-5551, 1994.
Chinnaiyan et al., Proc. Natl. Acad. Sci. USA, 97:1754-1759, 2000.
Christou et al., Proc. Natl. Acad. Sci. USA, 84(12):3962-3966, 1987.
Clark et al., Hum. Gene Ther., 6(10):1329-1341, 1995.
Cleary and Sklar, Proc. Natl. Acad. Sci. USA, (21):7439-7443, 1985.
Cleary et al., J. Exp. Med., 164(1):315-320, 1986.
Cocea, Biotechniques, 23(5):814-816, 1997.
Coffin, In: Virology, Fields et al. (Eds.), Raven Press, NY, 1437-1500, 1990.
Cong et al., Hum. Mol. Genet., 8:137-142, 1999.
Costa-Pereira and Cotter, Br. J. Cancer, 80:371-378, 1999.
Couch et al., Am. Rev. Resp. Dis., 88:394-403, 1963.
Counter et al., Oncogene, 16:1217-1222, 1998.
Coupar et al., Gene, 68:1-10, 1988.
Courey and Tjian, Cell, 55:887-898, 1988.
D'Halluin et al., Plant Cell, 4(12):1495-1505, 1992.
Dahm et al. J. Urol., 169:2220-2224, 2003.
Degliesposti et al., Immunity, 7:813-820, 1997.
Degliesposti et al., J. Experim. Med., 186:1165-1170, 1997.
Doehn and Jocham, Curr. Opin. Mol. Ther., 3:204-210, 2001.
EPO 0273085
Fechheimer, et al., Proc Natl. Acad. Sci. USA, 84:8463-8467, 1987.
Fieck et al., Nucleic Acids Res., 20(7):1785-1791, 1992.
Fillat et al., Curr. Gene Ther., 3:13-26, 2003.
Flotte and Carter, Gene Ther., 2(6):357-62, 1995.
Flotte et al., Am. J. Respir. Cell Mol. Biol., 7(3):349-356, 1992.
Flotte et al., Proc. Natl. Acad. Sci. USA, 90(22):10613-10617, 1993.
Fraley et al., Proc. Natl. Acad. Sci. USA, 76:3348-3352, 1979.
Franz et al., Cardioscience, 5(4):235-43, 1994.
Freundlieb et al., J. Gene Med., 1:4-12, 1999.
Friedmann, Science, 244:1275-1281, 1989.
Frost et al., Cell Immunol., 180:70-83, 1997.
Furth et al., Proc. Natl. Acad. Sci USA, 91:9302-9306, 1994.
Gash et al., Ann. Neurol., 44(3-Suppl):S121-125, 1998.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gibson et al., Molec. Cell. Biol., 20:205-212, 2000.
Giniger and Ptashne, Nature, 330:670-672, 1987.
Gliniak and Le, Cancer Res., 59:6153-6158, 1999.
Gomez-Foix et al., J. Biol. Chem., 267:25129-25134, 1992.
Gopal, Mol. Cell Biol., 5:1188-1190, 1985.
Gopal-Srivastava et al., J. Mol. Cell. Biol. 15(12):7081-90, 1995.
Gossen and Bujard, Proc. Natl. Acad. Sci. USA, 89:5547-5551, 1992.
Gossen et al., Science, 268: 1766-1769, 1995.
Graham and Prevec, Biotechnology, 20:363-390, 1992.
Graham and Van Der Eb, Virology, 52:456-467, 1973.
Graham et al., J. General Virology, 36:59-74, 1977.
Greenberg et al., Mol. Endocrinol., 8(2):230-239, 1994.
Greenberg et al., Proc. Natl. Acad. Sci. USA, 92(8):3439-3443, 1995.
Griffith and Lynch, Curr. Opin. Immun., 10:559-563, 1998.
Griffith and Reed, Science, 281:1309-1312, 1998.
Griffith et al., J. Immun., 165:2886-2894, 2000.
Griffith et al., J. Immunol., 161:2833-2840, 1998.
Griffith et al., Science, 270:1189-1192, 1995.
Grunhaus and Horwitz, Seminar in Virology, 3:237-252, 1992.
Hagio et al., Plant Cell Rep., 10(5):260-264, 1991.
Hahn et al., Nature, 400:464-468, 1999.
Hara et al., J. Urol., 169:2045-2048, 2003.
Harland and Weintraub, J. Cell Biol., 101(3):1094-1099, 1985.
Harrington et al., Gene Dev., 11:3109-3115, 1997.
Hauck and Stanners, J. Biol. Chem., 270:3602-3610, 1995.
Haviv and Curiel, Adv. Drug Deliv. Rev., 53:135-154, 2001.
He et al., Plant Cell Reports, 14 (2-3):192-196, 1994.
Hedlund et al., Cell Death Differ., 6:175-182, 1999.
Hedlund et al., Prostate, 36:92-101, 1998.
Heideman et al., Cancer Gene Ther., 8:342-351, 2001.
Henning et al., Hum. Gene Ther., 13:1427-1439, 2002.
Hensgens et al., Plant Mol. Biol., 22(6):1101-1127, 1993.
Hermonat and Muzycska, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Hillen and Wissmann, In: Protein-Nucleic Acid Interaction, Saeger and Heinemann (Eds.), Macmillan, London, 10:143-162, 1989.
Hinoda et al., Japanese J. Clin. Oncol., 21:75-81, 1991.
Hollstein et al., Science, 253(5015):49-53, 1991.
Horikawa et al., Cancer Res., 59:826-830, 1999.
Horwich et al. J. Virol., 64:642-650, 1990.
Hou and Lin, Plant Physiology, 111:166, 1996.
Howe, Jr. et al., J. Biol. Chem., 270:14168-14174, 1995.
Hussussian et al., Nat. Genet., 8(1): 15-21, 1994.
Hyer et al., Mol. Ther., 2:348-358, 2000.
Hyer et al., Mol. Ther., 2:348-358, 2000.
Hyer et al., Mol. Ther., 2:348-358, 2000.
Inouye and Inouye, Nucleic Acids Res., 13: 3101-3109, 1985.
Jeremias and Debatin, Europ. Cytokine Network, 9:687-688, 1998.
Jeremias et al., Blood, 91:4624-4631, 1998.
Jessup and Thomas, Cancer and Metastasis Rev., 8:263-280, 1989.
Jo et al., Nature Med., 6:564-567, 2000.
Jongmans et al., Urology, 62:559-565, 2003.
Kaeppler et al., Plant Cell Reports, 9:415-418, 1990.
Kamb et al., Nat. Genet., 8(1):23-26, 1994.
Kamb et al., Science, 2674:436-440, 1994.
Kanai, Mol. Biotechnol., 18:243-250, 2001.
Kaneda et al., Science, 243:375-378, 1989.
Kang, Mov. Disord., 13:(1)1:59-72, 1998
Kaplitt et al., Nat Genet., 8(2):148-54, 1994.
Kasahara et al., Science, 266(5189):1373-6, 1994.
Kashentseva et al., Cancer Res., 62:609-616, 2002.
Kasper et al., J. Mol. Endocrinol., 22:313-325, 1999.
Kato et al., J. Biol. Chem., 266:3361-3364, 1991.
Kayagaki et al., J. Exp. Med., 192:1777-1783, 1995.
Keane et al., Cancer Res., 59:734-741, 1999.
Kelly et al., J. Cell Biol., 129(2):383-96, 1995.
Kerr et al., Br. J. Cancer, 26(4):239-257, 1972.
Kijima et al., Cancer Res., 59:4906-4911, 1999.
Kilian et al., Hum. Mol. Genet., 12:2011-2019, 1997.
Kim et al., Clin. Cancer Res., 6:335-346, 2000.
Kimura et al., Dev. Growth Differ. 39(3):257-65, 1997.
Kim et al., Trends Mol. Med., 8:S68-73, 2002.
Kirschner-Hermanns and Jakse, Crit. Rev. Oncol. Hematol., 43:141-151, 2002.
Kistner et al., Proc. Natl. Acad. Sci. USA, 93:10933-10938, 1996.
Klein et al., Curr. Treat. Options Oncol., 2:403-411, 2001.
Klein et al., Nature, 327:70-73, 1987.
Knittel et al., Plant Cell Reports, 14(2-3):81-86, 1994.
Komatsu et al., Cancer Gene Ther., 7:885-892, 2000.
Kotin et al., Proc. Natl. Acad. Sci. USA, 87(6):2211-2215, 1990.
Kuang et al., J. Biol. Chem., 275:25065-25068, 2000.
Kubo et al., Hum. Gene Ther., 14:227-241, 2003.
LaFace et al., Virology, 162(2):483-486, 1988.
LaPointe et al., Appl. Microbiol Biotechnol., 42(6):895-900, 1995.
LaPointe et al., J. Biol. Chem., 263(19):9075-8, 1988.
Lareyre et al., J. Biol. Chem., 274(12):8282-8290, 1999.
Latham et al., Cancer Res. 60(2):334-41, 2000.
Laughlin et al., J. Virol., 60(2):515-524, 1986.
Lazzeri, Methods Mol Biol, 49:95-106, 1995.
Le Gal La Salle et al., Science, 259:988-990, 1993.
Lebkowski et al., Mol. Cell. Biol., 8(10):3988-3996, 1988.
Lee et al., Biochem. Biophys. Res. Commun., 240(2):309-313, 1997.
Lee et al., Cancer Gene Ther., 7:1113-1119, 2000.
Lee et al., Environ. Mol. Mutagen., 13(1):54-59, 1989.
Levenson et al., Hum. Gene Ther., 9(8):1233-1236, 1998.
Leverkus et al., Cancer Res., 60:553-559, 2000.
Levrero et al., Gene, 101:195-202, 1991.
Liu et al., Clin. Cancer Res., 4:1803-1811, 1998.
Luo et al., Proc. Natl. Acad. Sci. USA, 93:8907-8912, 1996.
Macejak and Sarnow, Nature, 353:90-94, 1991.
Macfarlane et al., J. Biol. Chem., 272:25417-25420, 1997.
Magari et al., J. Clin. Invest., 100:2865-2872, 1997.
Maniatis, et al., Molecular Cloning, A Laboratory Manual, Cold Spring Harbor Press, Cold Spring Harbor, N.Y., 1990.
Mann et al., Cell, 33:153-159, 1983.
Mariani et al., Eur. J. Immunol., 25:2303-2307, 1995.
Markowitz et al., J. Virol., 62:1120-1124, 1988.
Martin-Lorenzo et al., J. Immunol., 163:1274-1281, 1999.
McCabe and Martinell, Bio-Technology, 11(5):596-598, 1993.
McCarty et al., J. Virol., 65(6):2936-2945, 1991.
McLaughin et al., J. Virol., 62(6):1963-1973, 1988.
Mermod et al., Cell, 58:741-753, 1989.
Meuleman and Mulders, Eur. Urol., 43:95-101, 2003.
Meyerson et al., Cell, 90:785-795, 1997.
Miller et al., Am. J. Clin. Oncol., 15(3):216-221, 1992.
Mitsades et al., Cancer Res., 60:4122-4129, 2000.
Mitsumoto et al., Science, 265:1107-1110, 1994.
Mori et al., Cancer Res., 54(13):3396-3397, 1994.
Moss et al., Biol. Chem., 271(49):31688-94, 1996.
Muzio et al., J. Biol. Chem., 272:2952-2956, 1997.
Muzyczka, Curr. Topics Microbiol. Immunol., 158:97-129, 1992.
Nabel et al., Science, 244(4910):1342-1344, 1989.
Nagane et al., Cancer Res., 60:847-853, 2000.
Nakamura et al., J. Virol., 77:2512-2521, 2003.
Nakamura et al., Science, 277:955-959, 1997.
Nakayama et al., Nature Genet., 18:65-68, 1998.
Nettlebeck et al., Int. J. Cancer, 108:136-145, 2004.
Neumaier et al., J. Biol. Chem., 263:3202-3207, 1988.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nishihara et al., Anticancer Res., 18:1521-1525, 1998.
No et al., Proc. Natl. Acad. Sci USA, 93: 3346-3351, 1996.
Nobri et al., Nature (London), 368:753-756, 1995.
Nomoto et al., Gene, 236(2):259-271, 1999.
Ohi et al., Gene, 89(2):279-282, 1990.
Oikawa et al. Biochem. Biophys. Res. Comm., 146:464-469, 1987.
Okamoto et al., Proc. Natl. Acad. Sci. USA, 91(23):11045-11049, 1994.
Omirulleh et al., Plant Mol. Biol., 21(3):415-28, 1993.
Orlow et al., Int. J. Oncol., 15(1):17-24, 1994.
Osaki et al., Cancer Res., 54:5258-5261, 1994.
Ottonello et al., J. Immunol., 162:3601-3606, 1999.
Pan et al., FEBS Lett., 424:41-45, 1998.
Pan et al., Science, 277:815-818, 1997.
Pasland et al., Virology, 67:242-248, 1975.
Paxton et al., Proc. Natl. Acad. Sci. USA, 84:920-924, 1987.
PCT Appln. WO 92/17598
PCT Appln. WO 94/09699
PCT Appln. WO 94/09699
PCT Appln. WO 95/06128
PCT Appln. WO 95/06128
Pelletier and Sonenberg, Nature, 334(6180):320-325, 1988.
Perales et al., Proc. Natl. Acad. Sci. USA, 91:4086-4090, 1994.
Potrykus et al., Mol. Gen. Genet., 199:183-188, 1985.
Potter et al., Proc. Natl. Acad. Sci. USA, 81:7161-7165, 1984.
Pound et al., Urol. Clin. North Am., 24:395-406, 1997.
Powell et al., Curr. Biol., 9:1441-1447, 1999.
Racher et al., Biotechnology Techniques, 9:169-174, 1995.
Ragot et al., Nature, 361:647-650, 1993.
Renan, Radiother. Oncol., 19:197-218, 1990.
Rennie et al., Molecular Endocrinology, 7:23-36, 1993.
Rhodes et al., Methods Mol. Biol., 55:121-131, 1995.
Rich et al., Hum. Gene Ther., 4:461-476, 1993.
Richards et al., Hum. Gene Ther., 6:881-893, 1995.
Ridgeway, In: Vectors: A survey of molecular cloning vectors and their uses, Rodriguez and Denhardt (Eds.), Stoneham: Butterworth, 467-492, 1988.
Rippe et al., Mol. Cell Biol., 10:689-695, 1990.
Ritala et al., Plant Mol. Biol., 24(2):317-325, 1994.
Robbins et al., Int. J. Cancer, 53:892-897, 1993.
Rokhlin et al., Cancer Res., 57:1758-1768, 1997.
Rosenfeld et al., Science, 252:431-434, 1991.
Rosenfeld, et al., Cell, 68:143-155, 1992.
Rouvier et al., J. Exp. Med., 177:195-200, 1993.
Roux et al., Proc. Natl. Acad. Sci. USA, 86:9079-9083, 1989.
Rubinchik et al., Cancer Gene Ther., 10:814-822, 2003
Rubinchik et al., Gene Ther., 7:875-885, 2000.
Rubinchik et al., Gene Ther., 8:247-253, 2001.
Rubinchik et al., In: Virus Vectors for Gene Therapy: Methods in Protocols, Humana Press, Inc., NJ, 2002.
Rubinchik et al., Methods Enzymol., 346:529-547, 2002.
Rubinchik et al., Mol. Ther., 4:416-426, 2001.
Samulski et al., EMBO J., 10:3941-3950, 1991.
Samulski et al., J. Virol., 63:3822-3828, 1989.
Sasaki et al., Br. J. Urol., 81:852-855, 1998.
Schneider et al., Immunity, 7:831-836, 1997.
Schrewe et al., Mol. Cell. Biol., 10:2738-2748, 1990.
Schuur et al., J. Biol. Chem., 271(12):7043-7051, 1996.
Seol et al., Cancer Gene Ther., 10:540-548, 2003.
Serrano et al., Nature, 366:704-707, 1993.
Serrano et al., Science, 267(5195):249-252, 1995.
Shelling and Smith, Gene Therapy, 1:165-169, 1994.
Shinoura et al., Cancer Gene Ther., 7:739-748, 2000.
Shirakawa et al., Mol. Urol., 4:73-82, 2000.
Singsit et al., Transgenic Res., 6(2):169-176, 1997.
Smith-Arica et al., Mol. Ther., 2:579-587, 2000.
Snoek et al., Prostate, 36:256-263, 1998.
Spencer et al., Science, 262:1019-1024, 1993.
Stadler et al., J. Immunol., 152:1127-1133, 1994.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Stuart et al., J. Clin. Invest., 99:396-402, 1997.
Suda and Nagata, J. Experim. Med., 179:873-879, 1994.
Sun et al., Oncogene, 19:4513-4522, 2000.
Takahashi et al., Intl. Immunol., 6:1567-1574, 1994.
Takakura et al., Cancer Res., 59:551-557, 1999.
Tanaka and Herr, Cell, 60:375-386, 1990.
Tanaka et al., Nat. Med., 4(1):31-36, 1998.
Temin, In: Gene Transfer, Kucherlapati (Ed.), NY, Plenum Press, 149-188, 1986.
Thomas and Hersey, J. Immunol., 161:2195-2200, 1998.
Thompson et al., J. Clin. Lab. Anal., 5:344-366, 1991.
Tomes et al., Plant. Mol. Biol., 14(2):261-268, 1990.
Top et al., J. Infect. Dis., 124:155-160, 1971.
Torbet et al., Crop Science, 38(1):226-231, 1998.
Torbet et al., Plant Cell Reports, 14(10):635-640, 1995.
Tratschin et al., Mol. Cell. Biol., 4:2072-2081, 1984.
Tratschin et al., Mol. Cell. Biol., 5:3258-3260, 1985.
Triezenberg et al., Genes Dev., 2(6):718-729, 1988.
Tsujimoto and Croce, Proc. Natl. Acad. Sci. USA, 83(14):5214-5218, 1986.
Tsujimoto et al., Science, 228(4706):1440-1443, 1985.
Tsukada et al., Plant Cell Physiol., 30(4)599-604, 1989.
Tsumaki et al., J. Biol. Chem., 273(36):22861-22864, 1998.
Tur-Kaspa et al., Mol. Cell Biol., 6:716-718, 1986.
Uslu et al., Clin. Cancer Res., 3:963-972, 1997.
van Beusechem et al., Gene Ther., 10:1982-1991, 2003.
Van Eck et al., Plant Cell Reports, 14(5):299-304, 1995.
Van Ophoven et al., Prostate Cancer Prost. Dis., 2:227-233, 1999.
Vigne et al., Gene Ther., 10:153-162, 2003.
Voelkel-Johnson et al., Cancer Gene Ther., 9:164-172, 2002.
Volk et al., Cancer Biol. Ther., 2:511-515, 2003.
Wagner et al., Proc. Natl. Acad. Sci. USA 87(9):3410-3414, 1990.
Walczak et al., Nat. Med., 5:157-163, 1999.
Walsh et al., J. Clin. Invest, 94:1440-1448, 1994.
Wang et al., Nature Biotech., 15: 239-243, 1997.
Wang, Proc. Natl. Acad. Sci. USA, 93:8180-8184, 1994.
Webster, Mult. Scler., 3:113-120, 1997
Wei et al., Gene Therapy, 1:261-268, 1994.
Weinberg, Science, 254(5035):1138-1146, 1991.
Weinrich et al., Nature Genet., 17:498-502, 1997.
Wesseling et al., Gene Ther., 8:969-976, 2001.
Wilder et al., Cancer Res., 59:410-413, 1999a,
Wilder et al., Gene Therapy, 6:57-62, 1999b.
Wildner, Curr. Opin. Mol. Ther., 5:351-361, 2003.
Wiley et al., Immunity, 3:673-682, 1995.
Wilson et al., Science, 244:1344-1346, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Wu et al., Biochem. Biophys. Res. Commun., 233(1):221-6, 1997.
Wu et al., Mech. Dev., 101:61-69, 2001.
Wyborski and Short, Nucleic Acids Res., 19(17):4647-4653, 1991.
Wyborski et al., Environ. Mol. Mutagen., 28(4):447-458, 1996.
Yamamoto et al., Gastroenterology, 125:1203-1218, 2003.
Yamanaka et al., Hepatology, 32:482-490, 2000.
Yamauchi-Takihara, et al., Proc. Natl. Acad. Sci. USA, 86(10):3504-3508, 1989.
Yang et al., J. Virol., 68:4847-4856, 1994.
Yang et al., Proc. Natl. Acad. Sci. USA, 87:9568-9572, 1990.
Yazawa et al., World J. Surg., 26:783-789, 2002.
Yoder et al., Blood, 82 (Supp.): 1:347A, 1994.
Yu et al., Cancer Res., 60:2384-2389, 2000.
Zhang et al., Cancer Res., 59:2747-2753, 1999.
Zhang et al., Endocrinology, 141:4698-4710, 2000.
Zhao-Emonet et al., Biochim. Biophys. Acta, 1442(2-3):109-119, 1998.
Zhou et al., Exp. Hematol, 21:928-933, 1993.
Zhou et al., J. Exp. Med., 179:1867-1875, 1994.
Zhou et al., Plant Cell Reports, 12(11):612-616, 1993.
Ziober and Kramer, J. Bio. Chem., 271(37):22915-22, 1996.

1. An expression vector comprising:
(a) a first expression cassette comprising a first coding region that encodes a transcriptional activating factor (TAF), said first coding region being positioned under the transcriptional control of a first promoter comprising:
(i) a tissue specific regulatory element (TSRE); and
(ii) a TAF binding site (TBS);
and
(b) a second expression cassette comprising a second coding region that encodes a selected polypeptide, said second coding region being positioned under the transcriptional control of a second promoter comprising:
(i) a TSRE and a TBS; or
(ii) a TBS.

2. The expression vector of claim 1, wherein said vector is a non-viral vector.

3. The expression vector of claim 2, wherein said non-viral vector is comprised within a lipid delivery vehicle.

4. The expression vector of claim 3, wherein said lipid delivery vehicle is a liposome.

5. The expression vector of claim 1, wherein said vector is a viral vector.

6. The expression vector of claim 5, wherein said viral vector is comprised within a viral particle.

7. The expression vector of claim 5, wherein said viral vector is an adenoviral vector, a retroviral vector, a herpesviral vector, a pox virus vector, a polyoma virus vector, an alpha virus vector, or an adeno-associate viral vector.

8. The expression vector of claim 5, wherein said viral vector is a replication-deficient viral vector.

9. The expression vector of claim 8, wherein said replication-deficient viral vector is an adenoviral vector.

10. The expression vector of claim 5, wherein said viral vector is a replication-competent or conditionally replication-competent viral vector.

11. The expression vector of claim 10, wherein said replication-competent or conditionally replication-competent viral vector is an adenoviral vector.

12. The expression vector of claim 1, wherein said TAF is an antibiotic-regulated TAF, a hormone-regulated TAF, an human immunodeficiency virus TAF, or a hepatocye TAF.

13. The expression vector of claim 1, wherein said TSRE is derived from an ARR2PB promoter, a probasin promoter, an osteocalcin promoter, a human kallikrein 2 promoter, a DD3 promoter, a Clara cell secretory protein promoter, a liver-type pyruvate kinase proximal promoter, an apoE promoter, an alcohol dehydrogenase 6 promoter, a MUC-1 promoter, a survivin promoter, a CCR5 promoter a PSA promoter, an AFP promoter, an albumin promoter, or a telomerase promoter.

14. The expression vector of claim 1, wherein said selected polypeptide is a therapeutic polypeptide.

15. The expression vector of claim 14, wherein said therapeutic polypeptide is an anti-cancer polypeptide.

16. The expression vector of claim 15, wherein said anti-cancer polypeptide is a tumor suppressor, and inducer of apoptosis, and cell cycle regulator, a toxin, or an inhibitor of angiogenesis.

17. The expression vector of claim 14, wherein said therapeutic polypeptide is a enzyme, a cytokine, a hormone, a tumor antigen, a human antigen or a pathogen antigen.

18. The expression vector of claim 1, wherein said selected polypeptide is essential for vector replication.

19. The expression vector of claim 18, wherein (a) said vector is an adenoviral vector, and said selected polypeptide is an E1 protein, and E2 protein, an E4 protein, a fiber capside protein, an adenovirus terminal binding protein, an adenovirus polymerase, or (b) said vector is a herpes simplex virus and said selected polypeptide is a herpes simplex virus early or late gene.

20. The expression vector of claim 1, further comprising:
(c) a third expression cassette comprising a third coding region that encodes a first transcriptional silencer (TSI), said third coding region being positioned under the transcriptional control a third promoter comprising:
(i) a TSRE; and
(ii) a TAB;
and
(d) a fourth expression cassette comprising a fourth coding region that encodes a second TSI, said fourth coding region being positioned under the transcriptional control of a fourth promoter that is negatively regulated by said first TSI,
wherein said first, second and third promoters are negatively regulated by said second TSI.

21. A method of expressing a selected polypeptide in a cell of interest comprising contacting said cell with an expression vector comprising:
(a) a first expression cassette comprising a first coding region that encodes a transcriptional activating factor (TAF), said first coding region being positioned under the transcriptional control of a first promoter comprising:
(i) a tissue specific regulatory element (TSRE); and
(ii) a TAF binding site (IBS);
and
(b) a second expression cassette comprising a second coding region that encodes a selected polypeptide, said second coding region being positioned under the transcriptional control of a second promoter comprising:
(i) a TSRE and a TBS; or
(ii) a TBS.

22. The method of claim 21, wherein said vector is a non-viral vector.

23. The method of claim 21, wherein said vector is a viral vector.

24. The method of claim 23, wherein said viral vector is an adenoviral vector, a retroviral vector, a herpesviral vector, a pox virus vector, a polyoma virus vector, an alpha virus vector or an adeno-associate viral vector.

25. The method of claim 23, wherein said viral vector is a replication-deficient viral vector.

26. The method of claim 23, wherein said viral vector is a replication-competent viral vector.

27. The method of claim 23, wherein said viral vector is a conditionally replication-competent viral vector.

28. The method of claim 21, wherein said TAF is an antibiotic-regulated TAF, a hormone-regulated TAF, an human immunodeficiency virus TAF, or a hepatocye TAF.

29. The method of claim 21, wherein said TSRE is derived from an ARR2PB promoter, a probasin promoter, an osteocalcin promoter, a human kallikrein 2 promoter, a DD3 promoter, a Clara cell secretory protein promoter, a liver-type pyravate kinase proximal promoter, an apoE promoter, an alcohol dehydrogenase 6 promoter, a MUC-1 promoter, a survivin promoter, a CCR5 promoter a PSA promoter, an AFP promoter, an albumin promoter, or a telomerase promoter.

30. The method of claim 21, wherein said expression vector further comprises:
(c) a third expression cassette comprising a third coding region that encodes a first transcriptional silencer (TSI), said third coding region being positioned under the transcriptional control a third promoter comprising:
(i) a TSRE; and
(ii) a TAB;
and
(d) a fourth expression cassette comprising a fourth coding region that encodes a second TSI, said fourth coding region being positioned under the transcriptional control of a fourth promoter that is negatively regulated by said first TSI,
wherein said first, second and third promoters are negatively regulated by said second TSI.

31. A method of treating cancer comprising administering to a subject having cancer an expression vector comprising:
(a) a first expression cassette comprising a first coding region that encodes a transcriptional activating factor (TAF), said first coding region being positioned under the transcriptional control of a first promoter comprising:
(i) a tissue specific regulatory element (TSRE); and
(ii) a TAF binding site (TBS);
and
(b) a second expression cassette comprising a second coding region that encodes an anti-cancer polypeptide, said second coding region being positioned under the transcriptional control of a second promoter comprising:
(i) a TSRE and a TBS; or
(ii) a TBS.

32. The method of claim 31, wherein said vector is a non-viral vector.

33. The method of claim 31, wherein said vector is a viral vector.

34. The method of claim 33, wherein said viral vector is an adenoviral vector, a retroviral vector, a herpesviral vector, a pox virus vector, a polyoma virus vector, an alpha virus vector or an adeno-associate viral vector.

35. The method of claim 33, wherein said viral vector is a replication-deficient viral vector.

36. The method of claim 33, wherein said viral vector is a replication-competent viral vector.

37. The method of claim 33, wherein said viral vector is a conditionally replication-competent viral vector.

38. The method of claim 31, wherein said TAF is an antibiotic-regulated TAF, a hormone-regulated TAF, an human immunodeficiency virus TAF, or a hepatocye TAF.

39. The method of claim 31, wherein said TSRE is derived from an ARR2PB promoter, a probasin promoter, an osteocalcin promoter, a human kallikrein 2 promoter, a DD3 promoter, a Clara cell secretory protein promoter, a liver-type pyruvate kinase proximal promoter, an apoE promoter, an alcohol dehydrogenase 6 promoter, a MUC-1 promoter, a survivin promoter, a CCR5 promoter a PSA promoter, an AFP promoter, an albumin promoter, or a telomerase promoter.

40. The method of claim 31, wherein said expression vector further comprises a selectable or screenable marker.

41. The method of claim 31, wherein said cancer is breast cancer, ovarian cancer, fallopian tube cancer, cervical cancer, uterine cancer, prostate cancer, testicular cancer, pancreactic cancer, colon cancer, bladder cancer, liver cancer, stomach cancer, lung cancer, lymphoid cancer, brain cancer, thyroid cancer, head & neck cancer, skin cancer or leukemia.

42. The method of claim 31, wherein said expression vector is administered more than once.

43. The method of claim 31, wherein said expression vector is administered intratumorally, into tumor vasculature, local to a tumor, regional to a tumor or systemically.

44. The method of claim 31, wherein said expression vector is administered intravenously, intraarterially, subcutaneously, intramuscularly or into a natural or artificial body cavity.

45. The method of claim 31, wherein said cancer is a recurrent cancer, a metastatic cancer or a drug resistant cancer.

46. The method of claim 31, further comprising administering to said subject one or more distinct cancer therapies.

47. The method of claim 46, wherein said one or more distinct cancer therapies are chemotherapy, radiotherapy, hormonal therapy, immunotherapy, cryotherapy, toxin therapy, surgery or a second gene therapy.

48. The method of claim 46, wherein said expression vector is provided to said subject at the same time as said distinct cancer therapy.

49. The method of claim 46, wherein said expression vector is provided to said subject before or after said distinct cancer therapy.

50. The method of claim 31, wherein said expression vector further comprises:
(c) a third expression cassette comprising a third coding region that encodes a first transcriptional silencer (TSI), said third coding region being positioned under the transcriptional control a third promoter comprising:
(i) a TSRE; and
(ii) a TAB;
and
(d) a fourth expression cassette comprising a fourth coding region that encodes a second TSI, said fourth coding region being positioned under the transcriptional control of a fourth promoter that is negatively regulated by said first TSI,
wherein said first, second and third promoters are negatively regulated by said second TSI.

51. An expression vector comprising:
(a) a first expression cassette comprising a first coding region that encodes a first transcriptional silencer (TSI), said first coding region being positioned under the transcriptional control of a first promoter comprising a TSI binding site (SBS) for a second TSI;
(b) a second expression cassette comprising a second coding region that encodes a transcriptional activating factor (TAF), said second coding region being positioned under the transcriptional control of a second promoter comprising a tissue specific regulatory element (TSRE);
(c) a third expression cassette comprising a third coding region that encodes said second TSI, said third coding region being positioned under the transcriptional control of a third promoter comprising a tissue specific regulatory element (TSRE);
and
(d) a fourth expression cassette comprising a fourth coding region that encodes a selected polypeptide, said fourth coding region being positioned under the transcriptional control of a fourth promoter comprising a TAF binding site.

52. A method of expressing a selected polypeptide in a cell of interest comprising contacting said cell with an expression vector comprising:
(a) a first expression cassette comprising a first coding region that encodes a first transcriptional silencer (TSI), said first coding region being positioned under the transcriptional control of a first promoter comprising a TSI binding site (SBS) for a second TSI;
(b) a second expression cassette comprising a second coding region that encodes a transcriptional activating factor (TAF), said second coding region being positioned under the transcriptional control of a second promoter comprising a tissue specific regulatory element (TSRE);
(c) a third expression cassette comprising a third coding region that encodes said second TSI, said third coding region being positioned under the transcriptional control of a third promoter comprising a tissue specific regulatory element (TSRE);
and
(d) a fourth expression cassette comprising a fourth coding region that encodes a selected polypeptide, said fourth coding region being positioned under the transcriptional control of a fourth promoter comprising a TAF binding site.

53. A method of treating cancer comprising administering to a subject having cancer an expression vector comprising:
(a) a first expression cassette comprising a first coding region that encodes a first transcriptional silencer (TSI), said first coding region being positioned under the transcriptional control of a first promoter comprising a TSI binding site (SBS) for a second TSI;
(b) a second expression cassette comprising a second coding region that encodes a transcriptional activating factor (TAF), said second coding region being positioned under the transcriptional control of a second promoter comprising a tissue specific regulatory element (TSRE);
(c) a third expression cassette comprising a third coding region that encodes said second TSI, said third coding region being positioned under the transcriptional control of a third promoter comprising a tissue specific regulatory element (TSRE);
and
(d) a fourth expression cassette comprising a fourth coding region that encodes an anti-cancer polypeptide, said fourth coding region being positioned under the transcriptional control of a fourth promoter comprising a TAF binding site.


SEQUENCE LISTING (11 sequences)

SEQ ID NO: 1 - DNA, 11288 bp, Homo sapiens
  CDS (10804)..(10938)
aagcttaaaacccaatggattgacaacatcaagagttggaacaagtggacatggagatgttacttgtggaaatttagatgtgttcagctatcgggcaggagaatctgtgtcaaattccagcatggttcagaagaatcaaaaagtgtcacagtccaaatgtgcaacagtgcaggggataaaactgtggtgcattcaaactgagggatattttggaacatgagaaaggaagggattgctgctgcacagaacatggatgatctcacacatagagttgaaagaaaggagtcaatcgcagaatagaaaatgatcactaattccacctctataaagtttccaagaggaaaacccaattctgctgctagagatcagaatggaggtgacctgtgccttgcaatggctgtgagggtcacgggagtgtcacttagtgcaggcaatgtgccgtatcttaatctgggcagggctttcatgagcacataggaatgcagacattactgctgtgttcattttacttcaccggaaaagaagaataaaatcagccgggcgcggtggctcacgcctgtaatcccagcactttagaaggctgaggtgggcagattacttgaggtcaggagttcaagaccaccctggccaatatggtgaaaccccggctctactaaaaatacaaaaattagctgggcatggtggtgcgcgcctgtaatcccagctactcgggaggctgaggctggacaattgcttggacccaggaagcagaggttgcagtgagccaagattgtgccactgcactccagcttgggcaacagagccagactctgtaaaaaaaaaaaaaaaaaaaaaaaaaagaaagaaagaaaaagaaaagaaagtataaaatctctttgggttaacaaaaaaagatccacaaaacaaacaccagctcttatcaaacttacacaactctgccagagaacaggaaacacaaatactcattaactcacttttgtggcaataaaaccttcatgtcaaaaggagaccaggacacaatgaggaagtaaaactgcaggccctacttgggtgcagagagggaaaatccacaaataaaacattaccagaaggagctaagatttactgcattgagttcattccccaggtatgcaaggtgattttaacacctgaaaatcaatcattgcctttactacatagacagattagctagaaaaaaattacaactagcagaacagaagcaatttggccttcctaaaattccacatcatatcatcatgatggagacagtgcagacgccaatgacaataaaaagagggacctccgtcacccggtaaacatgtccacacagctccagcaagcacccgtcttcccagtgaatcactgtaacctcccctttaatcagccccaggcaaggctgcctgcgatggccacacaggctccaacccgtgggcctcaacctcccgcagaggctctcctttggccaccccatggggagagcatgaggacagggcagagccctctgatgcccacacatggcaggagctgacgccagagccatgggggctggagagcagagctgctggggtcagagcttcctgaggacacccaggcctaagggaaggcagctccctggatgggggcaaccaggctccgggctccaacctcagagcccgcatgggaggagccagcactctaggcctttcctagggtgactctgaggggaccctgacacgacaggatcgctgaatgcacccgagatgaaggggccaccacgggaccctgctctcgtggcagatcaggagagagtgggacaccatgccaggcccccatggcatggctgcgactgacccaggccactcccctgcatgcatcagcctcggtaagtcacatgaccaagcccaggaccaatgtggaaggaaggaaacagcatcccctttagtgatggaacccaaggtcagtgcaaagagaggccatgagcagttaggaagggtggtccaacctacagcacaaaccatcatctatcataagtagaagccctgctccatgacccctgcatttaaataaacgtttgttaaatgagtcaaattccctcaccatgagagctcacctgtgtgtaggcccatcacacacacaaacacacacacacacacacacacacacacacacacacacagggaaagtgcaggatcctggacagcaccaggcaggcttcacaggcagagcaaacagcgtgaatgacccatgcagtgccctgggccccatcagctcagagaccctgtgagggctgagatggggctaggcaggggagagacttagagagggtggggcctccagggagggggctgcagggagctgggtactgccctccagggagggggctgcagggagctgggtactgccctccagggagggggctgcagggagctgggtactgccctccagggagggggctgcagggagctgggtactgccctccagggagggggctgcagggagctgggtactgccctccagggaggcaggagcactgttcccaacagagagcacatcttcctgcagcagctgcacagacacaggagcccccatgactgccctgggccagggtgtggattccaaatttcgtgccccattgggtgggacggaggttgaccgtgacatccaaggggcatctgtgattccaaacttaaactactgtgcctacaaaataggaaataaccctactttttctactatctcaaattccctaagcacaagctagcaccctttaaatcaggaagttcagtcactcctggggtcctcccatgcccccagtctgacttgcaggtgcacagggtggctgacatctgtccttgctcctcctcttggctcaactgccgcccctcctgggggtgactgatggtcaggacaagggatcctagagctggccccatgattgacaggaaggcaggacttggcctccattctgaagactaggggtgtcaagagagctgggcatcccacagagctgcacaagatgacgcggacagagggtgacacagggctcagggcttcagacgggtcgggaggctcagctgagagttcagggacagacctgaggagcctcagtgggaaaagaagcactgaagtgggaagttctggaatgttctggacaagcctgagtgctctaaggaaatgctcccaccccgatgtagcctgcagcactggacggtctgtgtacctccccgctgcccatcctctcacagcccccgcctctagggacacaactcctgccctaacatgcatctttcctgtctcattccacacaaaagggcctctggggtccctgttctgcattgcaaggagtggaggtcacgttcccacagaccacccagcaacagggtcctatggaggtgcggtcaggaggatcacacgtccccccatgcccaggggactgactctgggggtgatggattggcctggaggccactggtcccctctgtccctgaggggaatctgcaccctggaggctgccacatccctcctgattctttcagctgagggcccttcttgaaatcccagggaggactcaacccccactgggaaaggcccagtgtggacggttccacagcagcccagctaaggcccttggacacagatcctgagtgagagaacctttagggacacaggtgcacggccatgtccccagtgcccacacagagcaggggcatctggaccctgagtgtgtagctcccgcgactgaacccagcccttccccaatgacgtgacccctggggtggctccaggtctccagtccatgccaccaaaatctccagattgagggtcctcccttgagtccctgatgcctgtccaggagctgccccctgagcaaatctagagtgcagagggctgggattgtggcagtaaaagcagccacatttgtctcaggaaggaaagggaggacatgagctccaggaagggcgatggcgtcctctagtgggcgcctcctgttaatgagcaaaaaggggccaggagagttgagagatcagggctggccttggactaaggctcagatggagaggactgaggtgcaaagagggggctgaagtaggggagtggtcgggagagatgggaggagcaggtaaggggaagccccagggaggccgggggagggtacagcagagctctccactcctcagcattgacatttggggtggtcgtgctagtggggttctgtaagttgtagggtgttcagcaccatctggggactctacccactaaatgccagcaggactccctccccaagctctaacaaccaacaatgtctccagactttccaaatgtcccctggagagcaaaattgcttctggcagaatcactgatctacgtcagtctctaaaagtgactcatcagcgaaatccttcacctcttgggagaagaatcacaagtgtgagaggggtagaaactgcagacttcaaaatctttccaaaagagttttacttaatcagcagtttgatgtcccaggagaagatacatttagagtgtttagagttgatgccacatggctgcctgtacctcacagcaggagcagagtgggttttccaagggcctgtaaccacaactggaatgacactcactgggttacattacaaagtggaatgtggggaattctgtagactttgggaagggaaatgtatgacgtgagcccacagcctaaggcagtggacagtccactttgaggctctcaccatctaggagacatctcagccatgaacatagccacatctgtcattagaaaacatgttttattaagaggaaaaatctaggctagaagtgctttatgctcttttttctctttatgttcaaattcatatacttttagatcattccttaaagaagaatctatccccctaagtaaatgttatcactgactggatagtgttggtgtctcactcccaacccctgtgtggtgacagtgccctgcttccccagccctgggccctctctgattcctgagagctttgggtgctccttcattaggaggaagagaggaagggtgtttttaatattctcaccattcacccatccacctcttagacactgggaagaatcagttgcccactcttggatttgatcctcgaattaatgacctctatttctgtcccttgtccatttcaacaatgtgacaggcctaagaggtgccttctccatgtgatttttgaggagaaggttctcaagataagttttctcacacctctttgaattacctccacctgtgtccccatcaccattaccagcagcatttggaccctttttctgttagtcagatgctttccacctcttgagggtgtatactgtatgctctctacacaggaatatgcagaggaaatagaaaaagggaaatcgcattactattcagagagaagaagacctttatgtgaatgaatgagagtctaaaatcctaagagagcccatataaaattattaccagtgctaaaactacaaaagttacactaacagtaaactagaataataaaacatgcatcacagttgctggtaaagctaaatcagatatttttttcttagaaaaagcattccatgtgtgttgcagtgatgacaggagtgcccttcagtcaatatgctgcctgtaatttttgttccctggcagaatgtattgtcttttctccctttaaatcttaaatgcaaaactaaaggcagctcctgggccccctccccaaagtcagctgcctgcaaccagccccacgaagagcagaggcctgagcttccctggtcaaaatagggggctagggagcttaaccttgctcgataaagctgtgttcccagaatgtcgctcctgttcccaggggcaccagcctggagggtggtgagcctcactggtggcctgatgcttaccttgtgccctcacaccagtggtcactggaaccttgaacacttggctgtcgcccggatctgcagatgtcaagaacttctggaagtcaaattactgcccacttctccagggcagatacctgtgaacatccaaaaccatgccacagaaccctgcctggggtctacaacacatatggactgtgagcaccaagtccagccctgaatctgtgaccacctgccaagatgcccctaactgggatccaccaatcactgcacatggcaggcagcgaggcttggaggtgcttcgccacaaggcagccccaatttgctgggagtttcttggcacctggtagtggtgaggagccttgggaccctcaggattactccccttaagcatagtggggacccttctgcatccccagcaggtgccccgctcttcagagcctctctctctgaggtttacccagacccctgcaccaatgagaccatgctgaagcctcagagagagagatggagctttgaccaggagccgctcttccttgagggccagggcagggaaagcaggaggcagcaccaggagtgggaacaccagtgtctaagcccctgatgagaacagggtggtctctcccatatgcccataccaggcctgtgaacagaatcctccttctgcagtgacaatgtctgagaggacgacatgtttcccagcctaacgtgcagccatgcccatctacccactgcctactgcaggacagcaccaacccaggagctgggaagctgggagaagacatggaatacccatggcttctcaccttcctccagtccagtgggcaccatttatgcctaggacacccacctgccggccccaggctcttaagagttaggtcacctaggtgcctctgggaggccgaggcaggagaattgcttgaacccgggaggcagaggttgcagtgagccgagatcacaccactgcactccagcctgggtgacagaatgagactctgtctcaaaaaaaaagagaaagatagcatcagtggctaccaagggctaggggcaggggaaggtggagagttaatgattaatagtatgaagtttctatgtgagatgatgaaaatgttctggaaaaaaaaatatagtggtgaggatgtagaatattgtgaatataattaacggcatttaattgtacacttaacatgattaatgtggcatattttatcttatgtatttgactacatccaagaaacactgggagagggaaagcccaccatgtaaaatacacccaccctaatcagatagtcctcattgtacccaggtacaggcccctcatgacctgcacaggaataactaaggatttaaggacatgaggcttcccagccaactgcaggtgcacaacataaatgtatctgcaaacagactgagagtaaagctgggggcacaaacctcagcactgccaggacacacacccttctcgtggattctgactttatctgacccggcccactgtccagatcttgttgtgggattgggacaagggaggtcataaagcctgtccccagggcactctgtgtgagcacacgagacctccccacccccccaccgttaggtctccacacatagatctgaccattaggcattgtgaggaggactctagcgcgggctcagggatcacaccagagaatcaggtacagagaggaagacggggctcgaggagctgatggatgacacagagcagggttcctgcagtccacaggtccagctcaccctggtgtaggtgccccatccccctgatccaggcatccctgacacagctccctcccggagcctcctcccaggtgacacatcagggtccctcactcaagctgtccagagagggcagcaccttggacagcgcccaccccacttcactcttcctccctcacagggctcagggctcagggctcaagtctcagaacaaatggcagaggccagtgagcccagagatggtgacagggcaatgatccaggggcagctgcctgaaacgggagcaggtgaagccacagatgggagaagatggttcaggaagaaaaatccaggaatgggcaggagaggagaggaggacacaggctctgtggggctgcagcccaggatgggactaagtgtgaagacatctcagcaggtgaggccaggtcccatgaacagagaagcagctcccacctcccctgatgcacggacacacagagtgtgtggtgctgtgcccccagagtcgggctctcctgttctggtccccagggagtgagaagtgaggttgacttgtccctgctcctctctgctaccccaacattcaccttctcctcatgcccctctctctcaaatatgatttggatctatgtccccgcccaaatctcatgtcaaattgtaaaccccaatgttggaggtggggccttgtgagaagtgattggataatgcgggtggattttctgctttgatgctgtttctgtgatagagatctcacatgatctggttgtttaaaagtgtgtagcacctctcccctctctctctctctctcttactcatgctctgccatgtaagacgttcctgtttccccttcaccgtccagaatgattgtaagttttctgaggcctccccaggagcagaagccactatgcttcctgtacaactgcagaatgatgagcgaattaaacctcttttctttataaattacccagtctcaggtatttctttatagcaatgcgaggacagactaatacaatcttctactcccagatccccgcacacgcttagccccagacatcactgcccctgggagcatgcacagcgcagcctcctgccgacaaaagcaaagtcacaaaaggtgacaaaaatctgcatttggggacatctgattgtgaaagagggaggacagtacacttgtagccacagagactggggctcaccgagctgaaacctggtagcactttggcataacatgtgcatgacccgtgttcaatgtctagagatcagtgttgagtaaaacagcctggtctggggccgctgctgtccccacttccctcctgtccaccagagggcggcagagttcctcccaccctggagcctccccaggggctgctgacctccctcagccgggcccacagcccagcagggtccaccctcacccgggtcacctcggcccacgtcctcctcgccctccgagctcctcacacggactctgtcagctcctccctgcagcctatcggccgcccacctgaggcttgtcggccgcccacttgaggcctgtcggctgccctctgcaggcagctcctgtcccctacaccccctccttccccgggctcagctgaaagggcgtctcccagggcagctccctgtgatctccaggacagctcagtctctcacaggctccgacgccccctatgctgtcacctcacagccctgtcattaccattaactcctcagtcccatgaagttcactgagcgcctgtctcccggttacaggaaaactctgtgacagggaccacgtctgtcctgctctctgtggaatcccagggcccagcccagtgcctgacacggaacagatgctccataaatactggttaaatgtgtgggagatctctaaaaagaagcatatcacctccgtgtggcccccagcagtcagagtctgttccatgtggacacaggggcactggcaccagcatgggaggaggccagcaagtgcccgcggctgccccaggaatgaggcctcaacccccagagcttcagaagggaggacagaggcctgcagggaatagatcctccggcctgaccctgcagcctaatccagagttcagggtcagctcacaccacgtcgaccctggtcagcatccctagggcagttccagacaaggccggaggtctcctcttgccctccagggggtgacattgcacacagacatcactcaggaaacggattcccctggacaggaacctggctttgctaaggaagtggaggtggagcctggtttccatcccttgctccaacagacccttctgatctctcccacatacctgctctgttcctttctgggtcctatgaggaccctgttctgccaggggtccctgtgcaactccagactccctcctggtaccaccatggggaaggtggggtgatcacaggacagtcagcctcgcagagacagagaccacccaggactgtcagggagaacatggacaggccctgagccgcagctcagccaacagacacggagagggagggtccccctggagccttccccaaggacagcagagcccagagtcacccacctccctccaccacagtcctctctttccaggacacacaagacacctccccctccacatgcaggatctggggactcctgagacctctgggcctgggtctccatccctgggtcagtggcggggttggtggtactggagacagagggctggtccctccccagccaccacccagtgagcctttttctagcccccagagccacctctgtcaccttcctgttgggcatcatcccaccttcccagagccctggagagcatggggagacccgggaccctgctgggtttctctgtcacaaaggaaaataatccccctggtgtgacagacccaaggacagaacacagcagaggtcagcactggggaagacaggttgtcctcccaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacaggtgaagggaggacaacctgggagagggtgggaggagggagctggggtctcctgggtaggacagggctgtgagacggacagagggctcctgttggagcctgaatagggaagaggacatcagagagggacaggagtcacaccagaaaaatcaaattgaactggaattggaaaggggcaggaaaacctcaagagttctattttcctagttaattgtcactggccactacgtttttaaaaatcataataactgcatcagatgacactttaaataaaaacataaccagggcatgaaacactgtcctcatccgcctaccgcggacattggaaaataagccccaggctgtggagggccctgggaaccctcatgaactcatccacaggaatctgcagcctgtcccaggcactggggtgcaaccaagatc

SEQ ID NO: 2 - protein, 44 aa, Homo sapiens
MESPSAPPHRWCIPWQRLLLTGEGRTTWERVGGGSWGLLGRTGL

SEQ ID NO: 3 - DNA, 5491 bp, Homo sapiens
  CDS (3415)..(3633)
gatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaggctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcaagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggtggggttcaccatgttggccaggctggtctcgaacttctgacctcagatgatccacctgcctctgcctcctaaagtgctgggattacaggtgtgagccaccatgcccagctcagaatttactctgtttagaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcaatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacctgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgcccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgttgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccatcctgactggcgctcggaggctcgtggagaccatctttctgggttccagccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcacccctggcaggtgtacggcttcgtgcgggcctgcctgcgccggctggtgcccccaggcctctggggctccaggcacaacgacgccgcttcctcaggaacaccaagaagttcatctccctggggaagcatgccaagctctcgctgcaggagctgacgtggaagatgagcgtgcgggactgcgcttggctgcgcaggagcccaggtgaggaggtggtggccgtcgagggcccagccccagagctgaatgcagtaggggctcaaaaagggggcaggcagagccctggtcctcctgtctccatcgtcacgtgggcacacgtggcttttcgctcaggacgtcgagtggacacggtgatctctgcctctgctctccctcctgtccagtttgcataaacttacgaggttcaccttcacgttttgatggacacgcggtttccaggcgccgaggccaagcagtgacagaggaggctgggcgcggcagtggagccgggttgccggcaatggggaaagtgtctggaagcacagacgctctggcgagggtgcctgcaggttacctataatcctcttcgcaatttcaagggtgggaatgagaggtggggacgagaacc

SEQ ID NO: 4 - protein, 73 aa, Homo sapiens
MPRAPRCRAVRSLLRSHYREVLPLATFVRRLGPQGWRLVQRGDPAAFRALVAQCLVCVPWDARPPPAAPSFRQ

SEQ ID NO: 5 - DNA, 4356 bp, Homo sapiens
  CDS (3997)..(4215)
gaattcacgtgactacgcacatcatgtacacactcccgtccacgaccgacccccgctgttttattttaatagctacaaagcagggaaatccctgctaaaatgtcctttaacaaactggttaaacaaacgggtccatccgcacggtggacagttcctcacagtgaagaggaacatgccgtttataaagcctgcaggcatctcaagggaattacgctgagtcaaaactgccacctccatgggatacgtacgcaacatgctcaaaaagaaagattttcaccccatggcaggggagtggttgggggttaaggacggtgggggcagcagctgggggctactgcacgcaccttttactaaagccagtttcctggttctgatggtattggctcagttatgggagactaaccataggggagtggggatgggggaacccggaggctgtgccatctttgccatgcccgagtgtcctgggcaggataatgctctagagatgcccacgtcctgattcccccaaacctgtggacagaacccgcccggccccagggcctttgcaggtgtgatctccgtgaggaccctgaggtctgggatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaggctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcgagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggggtggggttcaccatgttggccaggctggtctcgaacttctgacctcagatgatccacctgcctctgcctcctaaagtgctgggattacaggtgtgagccaccatgcccagctcagaatttactctgtttgaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcgatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacccgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgc

SEQ ID NO: 6 - protein, 73 aa, Homo sapiens; identical to SEQ ID NO: 4

SEQ ID NO: 7 - DNA, 16 bp, Artificial Sequence
  Description of Artificial Sequence  Synthetic Primer
  modified_base (7)..(9): N = A, C, G or T/U
ggtacannntgttcct

SEQ ID NO: 8 - DNA, 21 bp, Artificial Sequence
  Description of Artificial Sequence  Synthetic Primer
gcaagggcgaggagctgttca

SEQ ID NO: 9 - DNA, 24 bp, Artificial Sequence
  Description of Artificial Sequence  Synthetic Primer
aagttcaccttgatgccgttcttc

SEQ ID NO: 10 - DNA, 19 bp, Artificial Sequence
  Description of Artificial Sequence  Synthetic Primer
gtggggcgccccaggcacc

SEQ ID NO: 11 - DNA, 24 bp, Artificial Sequence
  Description of Artificial Sequence  Synthetic Primer
ctccttaatgtcacgcacgatttc